(19) United States
(12) Patent Application Publication
(10) Pub. No.: US 2007/0213393 A1
(43) Pub. Date: Sep. 13, 2007

(54) COMPOSITIONS AND METHODS FOR TREATING INFLAMMATORY CONDITIONS UTILIZING PROTEIN OR POLYSACCHARIDE CONTAINING ANTI-MICROTUBULE AGENTS

(75) Inventors: William L. Hunter, Vancouver (CA); David M. Gravett, Vancouver (CA); Richard T. Liggins, Coquitlam (CA); Philip M. Toleikis, Vancouver (CA)

Correspondence Address: SEED INTELLECTUAL PROPERTY LAW GROUP PLLC, 701 FIFTH AVENUE, SUITE 5400, SEATTLE, WA, 98104-7092, US

(73) Assignee: ANGIOTECH INTERNATIONAL AG, Zug (CH)

(21) Appl. No.: 11/687,528

(22) Filed: Mar. 16, 2007

Related U.S. Application Data
(63) Continuation of application No. 10/289,150, filed on Nov. 6, 2002.
Application No. 10/289,150 is a continuation-in-part of application No. 10/137,736, filed on May 1, 2002.
(60) Provisional application No. 60/288,017, filed on May 1, 2001.

(51) Int. Cl. A61K 31/337; A61K 31/165; A61K 47/36; A61K 47/42; C07C 233/01; C07D 305/14

(52) U.S. Cl. 514/449; 514/629; 514/773; 514/777; 549/510; 564/222

(57) ABSTRACT

Disclosed herein are compositions and methods for treating a variety of inflammatory conditions (e.g., inflammatory arthritis, adhesions, tumor excision sites, and fibroproliferative diseases of the eye). For example, there is provided a composition comprising a protein or polysaccharide containing dispersed (e.g., in micelle or liposome form) anti-microtubule agent, which may be formulated for administration to a patient in need thereof.

### CROSS-REFERENCE TO RELATED APPLICATION

This application is a continuation of U.S. application Ser. No. 10/289,150 filed Nov. 6, 2002, now pending; which is a continuation-in-part of U.S. patent application Ser. No. 10/137,736 filed May 1, 2002, now pending; which claims the benefit under 35 USC 119(e) of U.S. Provisional Application No. 60/288,017 filed May 1, 2001; all of these applications are incorporated herein by reference in their entireties.

### BACKGROUND OF THE INVENTION

1. Field of the Invention

The present invention relates generally to pharmaceutical compositions and methods, and more specifically, to compositions and methods for treating various inflammatory conditions or diseases (e.g., arthritis, including rheumatoid arthritis and osteoarthritis) utilizing a protein or polysaccharide combined with an anti-microtubule agent.

2. Description of the Related Art

Inflammatory conditions, whether of a chronic or acute nature, represent a substantial problem in the healthcare industry. Briefly, chronic inflammation is considered to be inflammation of a prolonged duration (weeks or months) in which active inflammation, tissue destruction and attempts at healing are proceeding simultaneously (Robbins Pathological Basis of Disease by R. S. Cotran, V. Kumar, and S. L. Robbins, W. B. Saunders Co., p. 75, 1989). Although chronic inflammation can follow an acute inflammatory episode, it can also begin as an insidious process that progresses with time, for example, as a result of a persistent infection (e.g., tuberculosis, syphilis, fungal infection) that causes a delayed hypersensitivity reaction, prolonged exposure to endogenous (e.g., elevated plasma lipids) or exogenous (e.g., silica, asbestos, cigarette tar, surgical sutures) toxins, or autoimmune reactions against the body's own tissues (e.g., rheumatoid arthritis, systemic lupus erythematosus, multiple sclerosis, psoriasis).

Inflammatory arthritis is a serious health problem in developed countries, particularly given the increasing number of aged individuals. For example, one form of inflammatory arthritis, rheumatoid arthritis (RA) is a multisystem chronic, relapsing, inflammatory disease affecting 1 to 2% of the world's population.

Although many organs can be affected, RA is basically a severe form of chronic synovitis that sometimes leads to destruction and ankylosis of affected joints (Robbins Pathological Basis of Disease, by R. S. Cotran, V. Kumar, and S. L. Robbins, W.B. Saunders Co., 1989). Pathologically the disease is characterized by a marked thickening of the synovial membrane which forms villous projections that extend into the joint space, multilayering of the synoviocyte lining (synoviocyte proliferation), infiltration of the synovial membrane with white blood cells (macrophages, lymphocytes, plasma cells, and lymphoid follicles; called an “inflammatory synovitis”), and deposition of fibrin with cellular necrosis within the synovium. The tissue formed as a result of this process is called pannus and eventually the pannus grows to fill the joint space. The pannus develops an extensive network of new blood vessels through the process of angiogenesis, which is essential to the evolution of the synovitis. Release of digestive enzymes (matrix metalloproteinases (e.g., collagenase, stromelysin)), and other mediators of the inflammatory process (e.g., hydrogen peroxide, superoxides, lysosomal enzymes, and products of arachadonic acid metabolism), from the cells of the pannus tissue leads to the progressive destruction of the cartilage tissue. The pannus invades the articular cartilage leading to erosions and fragmentation of the cartilage tissue. Eventually there is erosion of the subchondral bone with fibrous ankylosis, and ultimately bony ankylosis, of the involved joint.

It is generally believed, but not conclusively proven, that RA is an autoimmune disease and that many different arthrogenic stimuli activate the immune response in an immunogenetically susceptible host. Both exogenous infectious agents (Epstein-Barr virus, rubella virus, cytomegalovirus, herpes virus, human T-cell lymphotropic virus, Mycoplasma, and others) and endogenous proteins (collagen, proteoglycans, altered immunoglobulins) have been implicated as a causative agent that triggers an inappropriate host immune response. Regardless of the inciting agent, autoimmunity plays a role in the progression of the disease. In particular, the relevant antigen is ingested by antigen-presenting cells (macrophages or dendritic cells in the synovial membrane), processed, and presented to T lymphocytes. The T cells initiate a cellular immune response and stimulate the proliferation and differentiation of B lymphocytes into plasma cells. The end result is the production of an excessive inappropriate immune response directed against the host tissues (e.g., antibodies directed against type II collagen, antibodies directed against the Fc portion of autologous IgG (called “Rheumatoid Factor”)). This further amplifies the immune response and hastens the destruction of the cartilage tissue. Once this cascade is initiated numerous mediators of cartilage destruction are responsible for the progression of rheumatoid arthritis.

People with advanced rheumatoid arthritis have a mortality rate greater than some forms of cancer and because of this, treatment regimes have shifted towards aggressive early drug therapy designed to reduce the probability of irreversible joint damage. Recent recommendations of the American College of Rheumatology (Arthritis and Rheumatism 39(5):713-722, 1996) include early initiation of disease-modifying anti-rheumatic drug (DMARD) therapy for any patient with an established diagnosis and ongoing symptoms. Anticancer drugs have become the first line therapy for the vast majority of patients, with the chemotherapeutic drug methotrexate being the drug of choice for 60 to 70% of rheumatologists. The severity of the disease often warrants indefinite weekly treatment with this drug, and in those patients whose disease progresses despite methotrexate therapy (over 50% of patients), second line chemotherapeutic drugs such as cyclosporin and azathioprine (alone or in combination) are frequently employed.

The present invention discloses novel compositions, devices and methods for treating inflammatory conditions such as inflammatory arthritis, adhesions (e.g., surgical adhesions), fibroproliferative opthalmic conditions, and tumor excision sites, and further provides other related advantages.

### BRIEF SUMMARY OF THE INVENTION

Briefly stated, the present invention provides compositions and methods for the treatment of inflammatory conditions including, for example, inflammatory arthritis (e.g., rheumatoid arthritis, systemic lupus erythematosus, systemic sclerosis (scleroderma), mixed connective tissue disease, Sjögren's syndrome, ankylosing spondylitis, Behçet's syndrome, sarcoidosis, and osteoarthritis), adhesions (e.g., surgical adhesions), fibroproliferative opthalmic conditions, and tumor excision sites. The methods, compositions, and kits of the instant invention include pharmaceutically acceptable formulations of anti-microtubule agents (e.g., paclitaxel), wherein the anti-microtubule agent is dispersed by or in a carrier combined with a polysaccharide or polypeptide.

Within one aspect the invention provides a composition comprising a polypeptide or a polysaccharide and an anti-microtubule agent dispersed by a carrier. In another aspect, provided is a composition comprising a polypeptide or a polysaccharide and an anti-microtubule agent dispersed by a carrier, the anti-microtubule agent being dispersed independent of the polypeptide or polysaccharide. In yet another aspect, a composition comprising an anti-microtubule agent, a carrier that enhances the dispersability of the anti-microtubule agent in an aqueous medium, and at least one of a polypeptide or a polysaccharide.

In certain embodiments, a carrier comprises at least one of a co-solvent solution, liposomes, micelles, liquid crystals, nanocrystals, nanoparticles, emulsions, microparticles, microspheres, nanospheres, nanocapsules, polymers or polymeric carriers, surfactants, suspending agents, complexing agents such as cyclodextrins or adsorbing molecules such as albumin, surface active particles, and chelating agents. In further embodiments, a polysaccharide comprises hyaluronic acid and derivatives thereof, dextran and derivatives thereof, cellulose and derivatives thereof (e.g., methylcellulose, hydroxy-propylcellulose, hydroxy-propylmethylcellulose, carboxymethylcellulose, cellulose acetate phthalate, cellulose acetate succinate, cellulose acetate butyrate, hydroxypropylmethyl-cellulose phthalate), chitosan and derivative thereof, β-glucan, arabinoxylans, carrageenans, pectin, glycogen, fucoidan, chondrotin, pentosan, keratan, alginate, cyclodextrins, and salts and derivatives, including esters and sulfates, thereof. In further embodiments, the polysaccharide is not a cyclodextrin. In yet further embodiments, a polypeptide comprises homopolymers of polyamino acids such as poly(L-glutamic acid), polypeptides, proteins, peptides, copolymers of polyamino acids, collagen, albumin, fibrin and gelatin. In certain embodiments, an anti-microtubule agent may be prepared as a molecular, a colloidal or a coarse dispersion. The dispersion may be a solution or suspension and may contain one or more further components (apart from the polypeptide or polysaccharide) that act as a carrier to solubilize or otherwise disperse the anti-microtubule agent. In further embodiments, an anti-microtubule agent comprises taxanes such as paclitaxel, discodermolide, colchicine, vinca alkaloids such as vinblastine or vincristine, and analogues or derivatives of any of these. In certain other embodiments, a composition is in a form of a solution, a gel, a hydrogel, a film, a paste, a cream, a spray, an ointment, a powder, or a wrap.

In certain embodiments, a carrier forms micelles in the anti-microtubule composition, wherein the micelles contain an anti-microtubule agent. In a further embodiment, the carrier that forms micelles comprises chitosan or derivative thereof, or an amphiphilic block copolymer. In other embodiments, the block copolymer comprises a polyester hydrophobic block and a polyether hydrophilic block copolymer, or the block copolymer comprises a hydrophilic polyether block and a hydrophobic polyether block. In yet other embodiments, the carrier that forms micelles comprises a biodegradable component. In still other embodiments, the micelles have an average diameter ranging from about 10 nm to about 200 nm, more preferably an average diameter ranging from about 15 nm to about 150 nm, and most preferably an average diameter ranging from about 20 nm to about 100 nm. In more embodiments, the carrier forms nanoparticles containing an anti-microtubule agent, wherein the nanoparticles may further be either nanospheres or nanocapsules.

In further embodiments, the carrier comprises a co-solvent, wherein the co-solvent is miscible with water at a concentration of at least 10% v/v in water, and the anti-microtubule agent is soluble in a mixture of water and the co-solvent. In some embodiments, the co-solvent is one or more of ethanol, glycerol, ethoxydiglycol, N-methylpyrrolidinone (NMP), polyethyelene glycol (PEG) or a PEG derivative with a molecular weight of up to about 750 g/mol, or dimethylsulfoxide, or is one or more of PEG 200, PEG 300, ethanol, ethoxydiglycol, and NMP. In other embodiments, the anti-microtubule agent is a taxane, discodermolide, colchicine, vinca alkaloids, and analogues or derivatives of any of these. In certain embodiments, the anti-microtubule agent comprises a taxane, wherein the taxane is paclitaxel or an analog or derivative thereof, or the taxane is paclitaxel.

Within one aspect of the present invention, a polypeptide or polysaccharide combined with an anti-microtubule agent dispersed by or in a carrier may be utilized as a therapeutic composition. Compositions of the present invention may be administered by a variety of routes, depending on the condition targeted for treatment. In certain embodiments, the route of administration comprises intraarticular, intraperitoneal, topical, intravenous, ocular, or to the resection margin of tumors.

Within another aspect of the present invention, compositions are provided comprising a polypeptide or polysaccharide and a solubilized anti-microtubule agent. Representative examples of polypeptides include albumin, gelatin, collagen, and fragment or derivatives thereof. Representative examples of polysaccharides include chitosan, dextran, cellulose, and hyaluronic acid. Representative examples of anti-microtubule agents include taxanes, vinca alkaloids, colchicine, and analogues and derivatives of any of these. Within certain embodiments of the invention the solubilized anti-microtubule agent is a nanoparticles, nanoshpere, nanocapsule, or micelle containing an anti-microtubule agent. In one embodiment, the carrier forms an oil-in-water type emulsion, the emulsion comprising a dispersed non-aqueous phase containing the anti-microtubule agent, and a continuous phase comprising water. In another embodiment, the non-aqueous phase of the emulsion comprises at least one of benzyl benzoate, tributyrin, triacetin, safflower oil and corn oil. In still another embodiment, the dispersed phase is in droplets comprising an average diameter of less than about 100 nm, more preferably less than about 200 nm, and most preferably less than about 300 nm. In certain embodiments, the emulsion may be a microemulsion.

Also provided is a process for making the compositions of the instant invention. In one embodiment, a process for forming a composition comprises (a) contacting an anti-microtubule agent with a carrier to form an anti-microtubule agent dispersed by a carrier, and (b) combining (a) with a polypeptide or a polysaccharide, thereby forming the composition. In another embodiment, a process for forming a composition comprises (a) combining a polypeptide or a polysaccharide with a carrier in an aqueous medium, and (b) adding an anti-microtubule agent to (a), thereby forming a composition wherein the anti-microtubule agent is dispersed by the carrier. In another embodiment, the polypeptide or polysaccharide is a polysaccharide as described herein and in anther embodiment the polypeptide or polysaccharide is a polypeptide as described herein. In still another embodiment, the process for forming a composition results in a carrier that forms micelles, the micelles containing an anti-microtubule agent. In another embodiment, the carrier that forms micelles comprises chitosan or derivative thereof, or an amphiphilic block copolymer. In certain embodiments, the block copolymer comprises a polyester hydrophobic block and a polyether hydrophilic block copolymer, or the block copolymer comprises a hydrophilic polyether block and a hydrophobic polyether block. In yet other embodiments, the carrier that forms micelles comprises a biodegradable component. In other embodiments, the micelles have an average diameter ranging from about 10 nm to about 200 nm, or an average diameter ranging from about 15 nm to about 150 nm, or an average diameter ranging from about 20 nm to about 100 nm. In still other embodiments, the carrier forms nanoparticles containing an anti-microtubule agent, wherein the nanoparticles may further be either nanospheres or nanocapsules. In still another embodiment, the carrier comprises a co-solvent, wherein the co-solvent is miscible with water at a concentration of at least 10% v/v in water, and the anti-microtubule agent is soluble in a mixture of water and the co-solvent.

In further embodiments, the co-solvent is one or more of ethanol, glycerol, ethoxydiglycol, N-methylpyrrolidinone (NMP), polyethyelene glycol (PEG) or a PEG derivative with a molecular weight of up to about 750 g/mol, or dimethylsulfoxide, and more preferably is one or more of PEG 200, PEG 300, ethanol, ethoxydiglycol, and NMP. In another embodiment, the anti-microtubule agent is a taxane, discodermolide, colchicine, vinca alkaloids, and analogues or derivatives of any of these, or the anti-microtubule agent comprises a taxane, wherein the taxane is paclitaxel or an analog or derivative thereof, or the taxane is paclitaxel.

In other process embodiments, the polypeptide or polysaccharide is suspended or dissolved in an aqueous medium prior to combination with the dispersed anti-microtubule agent, which may be useful for forming a composition with the desired consistency, such as a gel or hydrogel. Preferably, the process of making a composition according to the instant invention is further sterilized by at least one of autoclaving, radiation, or filtering. In other embodiments, the compositions formed by the processes described herein are further lyophilized or spray dried. In addition, there is contemplated by the instant invention a composition produced by any of the aforementioned processes.

In another aspect, the invention provides kits, which may comprise one or more containers. In one aspect, the kit comprises an anti-microtubule agent dispersed by a carrier and a polysaccharide or a polypeptide. In a preferred embodiment, the kit comprises first container having an anti-microtubule agent dispersed by a carrier and a second container having a polysaccharide or a polypeptide. In a further preferred embodiment, the anti-microtubule agent dispersed by a carrier is in a form selected from the group consisting of a micelle, a nanoparticle, a microsphere, a liposome, an emulsion, a microemulsion, a cyclodextrin-complex, a co-solvent media, and a surfactant containing media, and most preferably a micelle. In another preferred embodiment, the polysaccharide or polypeptide is in the form of a solid, a liquid, a gel, or a hydrogel, and most preferably a hydrogel. In one aspect, the polypeptide or polysaccharide is a polypeptide selected from a polyamino acid homopolymer, a polyamino acid copolymer, a collagen, an albumin, a fibrin, a gelatin, and derivatives thereof. In another aspect, the polypeptide or polysaccharide is a polysaccharide selected from hyaluronic acid, hyaluronic acid derivatives, cellulose, cellulose derivatives, chitosan, chitosan derivatives, dextran, and dextran derivatives, and most preferably is hyaluronic acid or a derivative thereof. In a more preferred embodiment, the anti-microtubule agent is paclitaxel or an analogue or derivative thereof, and most preferably is paclitaxel. In other aspects, the anti-microtubule agent is dispersed in an aqueous medium or at least one of the kit components is lyophilized or spray dried.

In another aspect, there is provided by the instant invention a method for treating an inflammatory condition, comprising administering to a patient in need thereof a therapeutically effective amount of a composition comprising an anti-microtubule agent composition as described herein. In a further aspect, the method comprises delivering an anti-microtubule agent to a target site, wherein the method comprises forming an anti-microtubule agent composition as described herein, and introducing the anti-microtubule agent composition into an aqueous environment, wherein a target site is in contact with the aqueous environment. In certain embodiments, an inflammatory condition treated with the above methods may be inflammatory arthritis, adhesions, tumor excision sites, fibroproliferative ocular conditions, and the like. In other embodiments, the composition used in the above methods is in a form selected from the group consisting of a gel, a hydrogel, a film, a paste, a cream, a spray, an ointment, or a wrap. In further embodiments, the above methods are used to administer the compositions described herein by a route selected from intraarticular, intraperitoneal, topical, intravenous, ocular, or to the resection margin of tumors. In more embodiments, the anti-microtubule agent used in the compositions of these methods is paclitaxel or an analog or derivative thereof, and most preferably is paclitaxel. In other embodiments, the above methods are used to administer the anti-microtubule compositions described herein to a patient in need thereof who is a mammal, and more preferably the mammal is a human, horse, or dog.

These and other aspects of the present invention will become evident upon reference to the following detailed description and attached drawings. In addition, various references are set forth herein which describe in more detail certain procedures, devices, or compositions, and are therefore incorporated by reference in their entirety.

### DETAILED DESCRIPTION OF THE INVENTION

Prior to setting forth the invention, it may be helpful to an understanding thereof to set forth definitions of certain terms that will be used hereinafter.

“Inflammatory Conditions” as used herein refers to any of a number of conditions or diseases which are characterized by vascular changes: edema and infiltration of neutrophils (e.g., acute inflammatory reactions); infiltration of tissues by mononuclear cells; tissue destruction by inflammatory cells, connective tissue cells and their cellular products; and attempts at repair by connective tissue replacement (e.g., chronic inflammatory reactions). Representative examples of such conditions include many common medical conditions such as inflammatory arthritis, restenosis, adhesions (e.g., surgical adhesions), fibroproliferative opthalmic conditions, and tumor excision sites.

“Inflammatory arthritis” refers to a number of inflammatory diseases that principally (although not solely) affect one or more joints. Representative examples of inflammatory arthritis include, but are not limited to, rheumatoid arthritis, systemic lupus erythematosus, systemic sclerosis (scleroderma), mixed connective tissue disease, Sjögren's syndrome, ankylosing spondylitis, Behçet's syndrome, sarcoidosis, and osteoarthritis.

“Anti-microtubule agent” should be understood to include any protein, peptide, chemical, or other molecule that impairs the function of microtubules, for example, through the prevention or stabilization of tubulin polymerization. A wide variety of methods may be utilized to determine the anti-microtubule activity of a particular compound including, for example, assays described by Smith et al. (Cancer Lett 79(2):213-219, 1994) and Mooberry et al., (Cancer Lett. 96(2):261-266, 1995). Representative examples of anti-microtubule agents include taxanes, cholchicine, discodermolide, vinca alkaloids (e.g., vinblastine and vincristine), as well as analogues and derivatives of any of these.

“Dispersed Anti-Microtubule Agent” refers to anti-microtubule agents that may be prepared as molecular, colloidal or coarse dispersions. A “dispersed anti-microtubule agent” may be a solution or a suspension, and may contain one or more components that act as a carrier to stably solubilize or otherwise disperse one or more anti-microtubule agents. For example, an anti-microtubule agent such as paclitaxel may be dispersed by or in a carrier taking the form of a micelle, a nanosphere, or a co-solvent solution.

As noted above, the present invention provides methods for treating or preventing a wide variety of inflammatory diseases, comprising administering to a patient in need thereof a protein or polysaccharide containing solubilized or dispersed anti-microtubule agent wherein the agent is dispersed by a carrier as described herein. Representative examples of inflammatory diseases that may be treated include, for example, inflammatory arthritis, restenosis, adhesions (e.g., surgical adhesions), fibroproliferative opthalmic conditions, and tumor excision sites. Any concentration ranges recited herein are to be understood to include concentrations of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. As used herein, the term “about” means ±10%.

Discussed in more detail below are (I) Anti-Microtubule Agents; (II) Anti-Microtubule Agent Compositions and Formulations; and (III) Clinical Applications of the compositions described herein.

I. Anti-Microtubule Agents

Briefly, a wide variety of anti-microtubule agents can be utilized within the context of the present invention. Representative examples of such anti-microtubule agents includes taxanes, colchicine, LY290181, glycine ethyl ester, aluminum fluoride, and CI 980 (Allen et al., Am. J. Physiol. 261(4 Pt. 1): L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), vinca alkaloids (e.g., vinblastine and vincristine), discodermolide (ter Haar et al., Biochemistry 35: 243-250, 1996), as well as analogues and derivatives of any of these (see also PCT/CA97/00910 (WO 98/24427), which as noted above is hereby incorporated by reference in its entirety, for a list of additional anti-microtubule agents). Such compounds can act by either depolymerizing microtubules (e.g., colchicine and vinblastine), or by stabilizing microtubule formation (e.g., taxanes in general, and paclitaxel in particular).

A. Paclitaxel, Analogues and Derivatives

Within one preferred embodiment of the invention, the anti-microtubule agent is paclitaxel, a compound that disrupts mitosis (M-phase) by binding to tubulin to form abnormal mitotic spindles, or an analogue or derivative thereof. Briefly, paclitaxel is a highly derivatized diterpenoid (Wani et al., J. Am. Chem. Soc. 93:2325, 1971), which has been obtained from the harvested and dried bark of Taxus brevifolia (Pacific Yew) and Taxomyces andreanae and Endophytic fungus of the Pacific Yew (Stierle et al., Science 60:214-216, 1993).

The utility of the anti-microtubule agent paclitaxel, as a component of the compositions that comprise part of this invention, is demonstrated by data from a series of in vitro and in vivo experiments. Paclitaxel inhibits neutrophil activation (Jackson et al., Immunol. 90:502-10, 1997), decreases T-cell response to stimuli, and inhibits T-cell function (Cao et al., J. Neuroimmunol. 108:103-11, 2000), prevents the proliferation of and induces apoptosis in synoviocytes (Hui et al., Arth. Rheum. 40:1073-84, 1997), inhibits AP-1 transcription activity via reduced AP-1 binding to DNA (Hui et al., Arth. Rheum. 41:869-76, 1998), inhibits collagen induced arthritis in an animal model (Brahn et al., Arth. Rheum. 37:839-45, 1994; Oliver et al., Cellular Immunol. 157:291-9, 1994) but is non-toxic to non-proliferating cells, such as normal chondrocytes and non-proliferating synoviocytes (Hui et al., Arth. Rheum. 40:1073-84, 1997).

“Paclitaxel” (which should be understood herein to include formulations, prodrugs, epimers, isomers, analogues and derivatives such as, for example, TAXOL®, TAXOTERE®, docetaxel, 10-deacetyl analogues of paclitaxel and 3′N-desbenzoyl-3′N-t-butoxy carbonyl analogues of paclitaxel) may be readily prepared utilizing techniques known to those skilled in the art (see, e.g., Schiff et al., Nature 277:665-667, 1979; Long and Fairchild, Cancer Research 54:4355-4361, 1994; Ringel and Horwitz, J Nat'l Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonoxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, protaxols (2′- and/or 7-O-ester derivatives), (2′- and/or 7-O-carbonate derivatives), fluoro taxols, 9-deoxotaxane, 9-deoxotaxol, 7-deoxy-9-deoxotaxol, 10-desacetoxy-7-deoxy-9-deoxotaxol, derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′-succinyltaxol; 2′-(beta-alanyl)-taxol); 2′-γ-aminobutyryltaxol formate; ethylene glycol derivatives of 2′-succinyltaxol; prodrugs or derivatives having amino acids attached at either or both of the 2′ and 7 positions (R₉ and R₃, respectively); 2′-glutaryltaxol; 2′-(N,N-dimethylglycyl) taxol; 2′-(2-(N,N-dimethylamino)propionyl)taxol; 2′-orthocarboxybenzoyl taxol; 2′-aliphatic carboxylic acid derivatives of taxol, prodrugs {2′-(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′,7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol® analogs with modified phenylisoserine side chains, taxotere, (N-debenzoyl-N-tert-(butoxycaronyl)-10-deacetyltaxol, and taxanes (e.g., cephalomannine, brevifoliol, yunantaxusin and taxusin), debenzoyl-2-acyl paclitaxel derivatives, benzoate paclitaxel derivatives, sulfonated 2′-acryloyltaxol; sulfonated 2′-O-acyl acid paclitaxel derivatives, C18-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, C7 taxane derivatives, C10 taxane derivatives, 2-debenzoyl and 2-acyl paclitaxel derivatives, taxane analogs bearing new C2 and C4 functional groups, n-acyl paclitaxel analogues, 10-deacetyl taxol B, and 10-deacetyl taxol, benzoate derivatives of taxol, 2-aroyl-4-acyl paclitaxel analogues, ortho-ester paclitaxel analogues, and deoxy paclitaxel and deoxy paclitaxel analogues.

In one aspect, the Anti-microtubule agent is a taxane having the formula (C1): 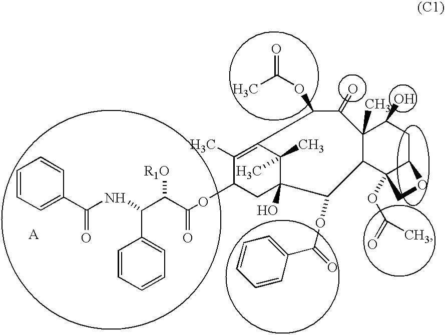
where the gray-highlighted portions may be substituted and the non-highlighted portion is the taxane core. A side-chain (labeled “A” in the diagram) is desirably present in order for the compound to have good activity as an Anti-microtubule agent. Examples of compounds having this structure include paclitaxel (Merck Index entry 7117), docetaxol (TAXOTERE®, Merck Index entry 3458), and 3′-desphenyl-3′-(4-ntirophenyl)-N-debenzoyl-N-(t-butoxycarbonyl)-10-deacetyltaxol.

In one aspect, suitable taxanes such as paclitaxel and its analogs and derivatives are disclosed in U.S. Pat. No. 5,440,056 as having the structure (C2): 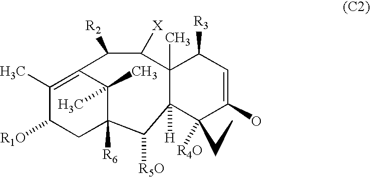
wherein X may be oxygen (paclitaxel), hydrogen (9-deoxotaxol or 9-deoxy derivatives, which may be further substituted to yield taxanes such as 7-deoxy-9-deoxotaxol, 10-desacetoxy-7-deoxy-9-deoxotaxol), thioacyl, or dihydroxyl precursors; R₁ is selected from paclitaxel or taxotere side chains or an alkanoyl of the formula (C3) 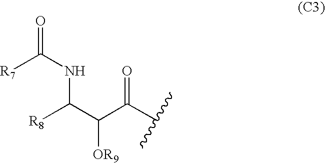
wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy to yield taxanes that include in some cases with further substitution: 10-deacetyltaxol, 10-desacetoxy-11,12-dihydrotaxol-10,12(18)-diene derivatives, 10-deacetyl taxol A, 10-deacetyl taxol B; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogs and derivatives useful as Anti-microtubule agents in the present invention are disclosed in PCT International Patent Application No. WO 93/10076. As disclosed in this publication, the analog or derivative should have a side chain attached to the taxane nucleus at C₁₃, as shown in the structure below (formula C4), in order to confer antitumor activity to the taxane.

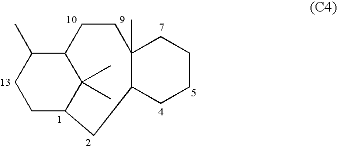

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, 10. As well, an oxetane ring may be attached at carbons 4 and 5. As well, an oxirane ring may be attached to the carbon labeled 4.

In one aspect, the taxane-based Anti-microtubule agent useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

B. Vinca Alkaloids

In another aspect, the Anti-microtubule agent is a Vinca Alkaloid. Vinca alkaloids have the following general structure. They are indole-dihydroindole dimers.

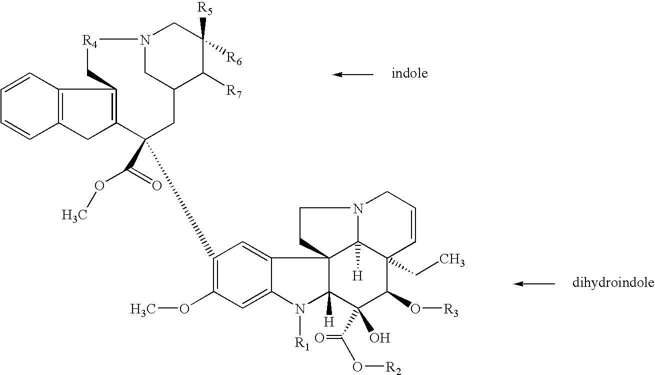

As disclosed in U.S. Pat. Nos. 4,841,045 and 5,030,620, R₁ can be a formyl or methyl group or alternately H. R₁ could also be an alkyl group or an aldehyde-substituted alkyl (e.g., CH₂CHO). R₂ is typically a CH₃ or NH₂ group. However it can be alternately substituted with a lower alkyl ester or the ester linking to the dihydroindole core may be substituted with C(O)—R where R is NH₂, an amino acid ester or a peptide ester. R₃ is typically C(O)CH₃, CH₃ or H. Alternately a protein fragment may be linked by a bifunctional group such as maleoyl amino acid. R₃ could also be substituted to form an alkyl ester, which may be further substituted. R₄ may be —CH₂— or a single bond. R₅ and R₆ may be H, OH or a lower alkyl, typically —CH₂CH₃. Alternatively R₆ and R₇ may together form an oxetane ring. R₇ may alternately be H. Further substitutions include molecules wherein methyl groups are substituted with other alkyl groups, and whereby unsaturated rings may be derivatized by the addition of a side group such as an alkane, alkene, alkyne, halogen, ester, amide or amino group.

Exemplary vinca alkaloids include without limitation vinblastine, vincristine, vincristine sulfate, vindesine, and vinorelbine, having the structures:

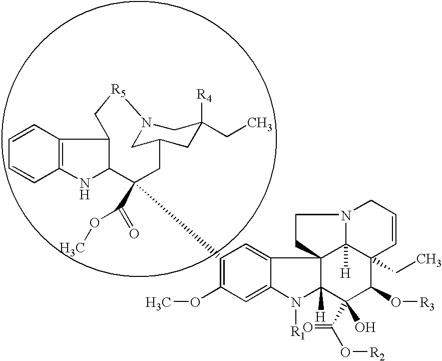

| | R₁ | R₂ | R₃ | R₄ | R₅ |
|---|---|---|---|---|---|
| Vinblastine: | CH₃ | CH₃ | C(O)CH₃ | OH | CH₂ |
| Vincristine: | CH₂O | CH₃ | C(O)CH₃ | OH | CH₂ |
| Vindesine: | CH₃ | NH₂ | H | OH | CH₂ |
| Vinorelbine: | CH₃ | CH₃ | CH₃ | H | single bond |

Analogs typically require the side group (shaded area) in order to have activity. Other exemplary vinca alkaloids useful in the compositions described herein include without limitation vinflunine (20′,20′-difluoro-3′,4′-dihydrovinorelbine), vinepidine, desformyl-vincristine, desacetyl-desformyl-vincristine, vinblastine sulfate and vindesine sulfate.

Vinca alkaloids act as anti-microtubule agents generally by inhibiting polymerization of microtubules.

II. Anti-Microtubule Agent Compositions and Formulations

As noted above, therapeutic anti-microtubule agents, preferably paclitaxel or an analogue or derivative thereof, may be formulated in a variety of manners for use in treating inflammatory conditions, as described herein. A variety of problems are associated with several current formulations of hydrophobic anti-microtubule agents, such as paclitaxel, which range from an unacceptable toxicity level to a failure to prevent rapid clearance of an anti-microtubule agent. The instant invention relates, generally, to the surprising discovery that anti-microtubule agents, and more specifically hydrophobic agents, may be formulated at clinically relevant concentrations to maximize in vivo stability, to maximize release half-life, and to increase efficacy against inflammatory diseases.

One advantage of the compositions described herein is that the compositions may be prepared by combining an anti-microtubule agent dispersed by at least one carrier with a polypeptide or polysaccharide. In a preferred embodiment, there is provided a composition comprising an anti-microtubule agent solubilized or dispersed by a carrier and a polypeptide or a polysaccharide, wherein the anti-microtubule agent is solubilized or dispersed independent of the polypeptide or polysaccharide. As used in the compositions and methods of the instant invention, the polypeptide or polysaccharide is capable of associating with, incorporating, holding, containing, carrying, occluding, absorbing, adsorbing, or encompassing an anti-microtubule agent in a dispersed for, or capable of functioning as a carrier to disperse an anti-microtubule agent. In a preferred embodiment, the polypeptide or polysaccharide of the compositions contemplated by the instant invention is not a carrier for dispersing an anti-microtubule agent.

A. Carriers

As used herein, a “carrier” is an agent that enhances the solubility or dispersability of an anti-microtubule agent in an aqueous medium (particularly a hydrophobic agent such as paclitaxel) or a non-aqueous medium. The anti-microtubule agent dispersed by a carrier may be prepared as molecular, colloidal or coarse dispersions. In certain embodiments, the anti-microtubule agent is water-solubilized in the sense that the anti-microtubule agent is dispersed or dissolved throughout an aqueous media. In certain preferred embodiments, an anti-microtubule agent remains dispersed or dissolved throughout the aqueous media even upon the addition of water to the composition. An anti-microtubule agent dispersed by a carrier may be in the form of a solution or of a suspension, and may contain one or more further components (e.g., polypeptide or polysaccharide) that may act as a second carrier or may act to solubilize or otherwise disperse the anti-microtubule agent.

Exemplary carriers may include one or more of the following: hydroxypropyl β-cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073); liposome/gel (WO 94/26254); nanocapsules (Bartoli et al., J. Microencapsulation 7(2):191-197, 1990), micelles (Alkan-Onyuksel et al., Pharm. Res. 11(2):206-212, 1994); implants (U.S. Pat. No. 4,882,168; Jampel et al., Invest. Ophthalm. Vis. Science 34(11): 3076-3083, 1993; Walter et al., Cancer Res. 54:22017-2212, 1994); nanoparticles (WO 01/89522; Violante and Lanzafame PAACR; U.S. Pat. No. 5,145,684; U.S. Pat. No. 5,399,363); nanospheres (Hagan et al., Proc. Intern. Symp. Control Rel. Bioact. Mater. 22, 1995; Kwon et al., Pharm Res. 12(2):192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994); an emulsion/solution (U.S. Pat. No. 5,407,683); surfactant micelles (U.S. Pat. No. 5,403,858); synthetic phospholipid compounds (U.S. Pat. No. 4,534,899), gas borne dispersion (U.S. Pat. No. 5,301,664); foam; spray; gel; lotion; cream; ointment; dispersed vesicles; particles or droplets; solid- or liquid-aerosols; microemulsions (U.S. Pat. No. 5,330,756), polymeric shell (nano- and micro-capsule) (U.S. Pat. No. 5,439,686), a surface-active agent (U.S. Pat. No. 5,438,072), and liquid emulsions (Tarr et al., Pharm Res. 4:62-165, 1987). Other exemplary carriers suitable for use in the compositions and methods described herein include co-solvents such as ethanol, liquid crystals, microparticles, microspheres, polymers or polymeric carriers, suspending agents, adsorbing agents such as albumin, surfactants, surface active particles, chelating agents, and the like. In addition, as provided herein and would be known in the art at the time of this invention, a wide variety of other carriers may be selected, such as polymers or non-polymeric molecules (see, e.g., WO 98/24427, which as noted above is hereby incorporated by reference in its entirety). In one aspect, the polysaccharides of the compositions contemplated by the invention do not include cyclodextrin. In certain other aspects, the composition comprises an anti-microtubule agent dispersed by a carrier and a polypeptide or polysaccharide, wherein the carrier is not a polypeptide or a polysaccharide. In certain embodiments, a composition of the present invention may include a first carrier material and a second carrier material.

Within preferred embodiments of the invention, the anti-microtubule agent is contained primarily within, or is generated to be, in a dispersed or solubilized form with a carrier. In one aspect, the anti-microtubule agents of the present invention are not readily water-soluble (i.e., have a hydrophobic character). An anti-microtubule agent dispersed by or in a carrier can include water soluble forms of an anti-microtubule agent, anti-microtubule agents contained within a liposome carrier, or anti-microtubule agents contained primarily within or generated to be in a carrier that forms a micelle (i.e., with a hydrophobic core and a hydrophilic exterior). Alternatively, the anti-microtubule agent can be dispersed with carriers such as ethoxydiglycol (Transcutol®), polyethylene glycol (e.g., PEG 200 or 300 or MePEG 350), N-methyl-pyrrolidone (NMP), ethanol, or surfactants (e.g., Tween® or Pluronic®). In one aspect, the compositions have a carrier that forms liposomes, wherein the liposomes comprise at least one of triolein, dipalmityl-phospatidylcholine, egg phosphotidylchloline, glycerol, polysorbate 80, and cholesterol.

In certain embodiments, there is provided a composition comprising a polypeptide or polysaccharide and an anti-microtubule agent dispersed by or in a carrier. An anti-microtubule agent may be solubilized in the presence of a carrier alone or, optionally, in the presence of other agents, including without limitation at least one polysaccharide, polypeptide, surfactant, preservative, water, and the like. In another preferred aspect, the invention pertains to a composition comprising a polypeptide or a polysaccharide and an anti-microtubule agent dispersed by a carrier, the anti-microtubule agent being dispersed independent of the polypeptide or polysaccharide. In certain aspects, the surfactant may be selected from polysorbate 80 (CAS Registry No. 9005-65-6), polysorbate 80 (glycol) (CAS Registry No. 9005-65-6); block copolymers of ethylene oxide and propylene oxide; lecithin; and sorbitan monopalmitate. In another embodiment, the compositions of this invention may further comprise water and/or have a pH of about 3-9. In yet another preferred aspect, the composition comprises an anti-microtubule agent, a carrier that enhances the dispersability of the anti-microtubule agent in an aqueous medium, and at least one of polypeptide or a polysaccharide.

B. Polypeptides and Polysaccharides

In certain embodiments of the instant invention, a polypeptide or polysaccharide may be combined with an anti-microtubule agent dispersed by a carrier. In another embodiment, a polypeptide or polysaccharide may be combined with an anti-microtubule agent dispersed by a carrier in an aqueous environment prior to addition of an anti-microtubule agent. For purposes of the instant invention, a polypeptide or a polysaccharide is a molecule capable of associating with, incorporating, holding, containing, carrying, occluding, absorbing, adsorbing, or encompassing another agent, such as a solubilized anti-microtubule agent. In one aspect, a polypeptide or a polysaccharide may function as a super carrier (i.e., a second carrier of the first carrier that disperses the anti-microtubule agent). In certain embodiments, a composition of the present invention may include a first carrier material and a second carrier material.

In another aspect, the polysaccharides and polypeptides of the instant invention can be fashioned to exhibit a variety of forms with desired release characteristics and/or with specific desired properties. For example, polymers can be formed into gels by dispersing them into a solvent such as water. In certain embodiments, polysaccharides and polypeptides and other polymers can be fashioned to release a therapeutic agent upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polysaccharides include carboxymethyl cellulose, cellulose acetate trimellilate, hydroxypropylmethylcellulose phthalate, hydroxypropylmethylcellulose acetate succinate, chitosan and alginates. Yet other pH sensitive polymers include any mixture of a pH sensitive polymer and a water soluble polymer. In one aspect, the polysaccharides and peptides of the invention may be pH sensitive.

Likewise, polysaccharides and polypeptides and other polymers can be fashioned to be temperature sensitive (see, e.g., Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B.V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermogelling polymers, in particular polysaccharides, include cellulose ether derivatives, such as hydroxypropyl cellulose, hydroxyethyl cellulose, methyl cellulose, and hydroxypropylmethyl cellulose.

As used herein, a “polysaccharide” means a combination of at least three monosaccharides that are generally joined by glycosidic bonds. Representative examples of suitable polysaccharides include hyaluronic acid, dextran, cellulose and derivatives thereof (e.g., methylcellulose, hydroxy-propylcellulose, hydroxy-propylmethylcellulose, carboxymethylcellulose, cellulose acetate phthalate, cellulose acetate succinate, hydroxypropylmethylcellulose phthalate), β-glucan, arabinoxylans, carrageenans, pectin, glycogen, fucoidan, chondrotin, pentosan, keratan, alginate and salts and derivatives, including esters and sulfates, thereof. In one aspect, the composition comprises a polysaccharide and an anti-microtubule agent dispersed by a carrier.

An exemplary polysaccharide includes without limitation hyaluronic acid (also known as hyaluronan) and derivatives thereof (see, e.g., U.S. Pat. Nos. 5,399,351, 5,266,563, 5,246,698, 5,143,724, 5,128,326, 5,099,013, 4,913,743, and 4,713,448), including esters, partial esters and salts of hyaluronic acid. Hyaluronic acid (HA) as used herein comprises an acidic polysaccharide of repeating subunits of D-glucuronic acid and N-acetyl-D-glucosamine, as well as salts and derivatives thereof. HA may be isolated from natural sources, such as rooster combs and human umbilical cord (it is also found in the vitreous of the human eye), or from certain bacteria as a highly polymerized mucopolysaccharide. Naturally occurring HA can be purified according to accepted procedures known to those having skill in the art at the time of this invention. HA may also be synthetically produced as crosslinked (e.g., hylan) or non-crosslinked HA.

Hylans are cross-linked hyaluronic acids with increased molecular weight and increased chemical and/or elastoviscous properties. Hylan (hylan fibers) can be prepared, for example, from HA prepared from rooster combs using formaldehyde as previously described (see U.S. Pat. No. 4,713,448). In addition, by way of example but not limitation, cross-linked derivates of hyaluronic acid also include those crosslinked with vinyl sulfone (see U.S. Pat. No. 4,605,691) or other polymers of low molecular weight (see U.S. Pat. No. 4,582,865). Such crosslinking may also be used to prepare hylan fibers. As used herein, crosslinking may be complete or partial.

Exemplary salts of HA include without limitation sodium hyaluronate, including those of alkali or alkaline earth metal salts, which may have a molecular weight ranging from 50,000 to 5×10⁶. Higher molecular weight HA may be used in the compositions of the instant invention, such as HA having a molecular weight between 8×10⁶ and 1.3×10⁷. Natural sources, such as rooster combs, may contain sodium HA of molecular weight of between 1×10⁶ and 4.5×10⁶, which may be degraded by heating to a molecular weight of 30,000-200,000. Methyl ester modified HA may also be in the compositions of the instant invention, which can be obtained by treatment of high molecular weight HA with, for example, diazomethane in ether (Jeanloz et al., J. Biol. Chem. 186:495-511, 1950).

Certain advantages of modified derivates of naturally occurring HA may include improved pharmacological and therapeutic properties, for example, stability and/or resistance to degradation by naturally occurring enzymes upon administration to a patient, such as a mammal (including humans, horses, and dogs). Typical esters of HA may be prepared using aliphatic, araliphatic, cycloaliphatic or etherocyclic alcohols, and the like. All or any portion of the available carboxylic of HA may be esterified. Ester modification can be used to decrease water solubility of HA. Also contemplated are hyaruronic acids containing mixed esters, for example, partial treatment with an aliphatic alcohol followed by treatment with an araliphatic alcohol, which may require an intermediate purification step known by those having skill in the art.

In certain embodiments, the compositions of the instant invention include a polysaccharide selected from hyaluronic acid, hyaluronic acid derivatives, cellulose, cellulose derivatives, chitosan, chitosan derivatives, dextran, and dextran derivatives. In a more preferred embodiment, the compositions of the instant invention include hyaluronic acid or derivatives thereof. In another preferred embodiment, the hyaluronic acid or derivative thereof is crosslinked (fully or partially). Another preferred embodiment comprises hyaluronic acid or a derivative thereof that is not crosslinked and has a viscosity average molecular weight in the range of about 50 kDa to about 6000 kDa, more preferably the viscosity average molecular weight of the hyaluronic acid or derivative thereof is greater than 800 kDa or greater than about 900 kDa. In a further preferred embodiment, the composition is in the form of a hydrogel, as described herein.

As used herein, “polypeptide” includes peptides, proteins, cyclic proteins, branched proteins, polyamino acids, and derivatives of each of these (including those with non-naturally occurring amino acids known in the art), which may be naturally or synthetically derived. An “isolated peptide, polypeptide, or protein” is an amino acid sequence that is essentially free from contaminating cellular components, such as carbohydrate, lipid, nucleic acid (DNA or RNA), or other proteinaceous impurities associated with the polypeptide in nature. Preferably, an isolated polypeptide is sufficiently pure for therapeutic use at a desired dose. Representative examples of polypeptides suitable for the compositions and methods of the present invention include homopolymers of polyamino acids such as poly(L-glutamic acid), copolymers of polyamino acids that include at least two different amino acids, polypeptides, proteins, peptides, collagen, albumin, fibrin and gelatin. In one aspect, the composition comprises a polypeptide and an anti-microtubule agent dispersed by a carrier. In a preferred embodiment, the polypeptide is a polyamino acid homopolymer, polyamino acid copolymer, collagen, albumin, fibrin, or gelatin.

C. Compositions, Methods of Making Same, and Kits

A wide variety of forms may be fashioned by the compositions of the present invention, including for example, rod-shaped devices, pellets, slabs, particulates, micelles, films, molds, sutures, threads, gels, creams, ointments, pastes, sprays, tablets, and capsules (see, e.g., Goodell et al., Am. J. Hosp. Pharm. 43:1454-1461, 1986; Langer et al., “Controlled release of macromolecules from polymers”, in Biomedical Polymers, Polymeric Materials and Pharmaceuticals for Biomedical Use, Goldberg, E. P., Nakagim, A. (eds.) Academic Press, pp. 113-137, 1980; Rhine et al., J. Pharm. Sci. 69:265-270, 1980; Brown et al., J. Pharm. Sci. 72:1181-1185, 1983; and Bawa et al., J. Controlled Release 1:259-267, 1985). Therapeutic agents may be linked by occlusion in the matrices of the polymer, bound by covalent linkages, or encapsulated in microcapsules. Within certain preferred embodiments of the invention, therapeutic compositions are provided in non-capsular or non-tablet formulations, such as particles (which may be spheres) ranging from nanometers to micrometers in size, pastes, threads or sutures of various size, films and sprays.

In certain embodiments, compositions of the present invention can be formed into a gel, a hydrogel, a film, a paste, a cream, a spray, an ointment, a powder, or a wrap. A gel is a semisolid characterized by relatively high yield values as described in Martin's Physical Pharmacy (Fourth Edition, Alfred Martin, Lea & Febiger, Philadelphia, 1993, pp 574-575). Gels possess properties such as elevated viscosity and elasticity, which may be reduced with increased dilution with an aqueous medium such as water. Gels may contain only non-crosslinked and/or partially crosslinked polymers. Alternately, polymers may be crosslinked to form systems that are herein defined as hydrogels. A hydrogel will maintain an elevated level of viscosity and elasticity when diluted with an aqueous solution, such as water. Crosslinking may be accomplished by several means including covalent, hydrogen, ionic, hydrophobic, chelation complexation, and the like. Gels may contain non-crosslinked, fully crosslinked, and partially crosslinked materials.

In addition to any of the compositions described herein, any pharmaceutically or veterinarily acceptable vehicle, carrier, diluent, or excipient, may be included along with, optionally, other components. Pharmaceutically or veterinarily acceptable excipients for therapeutic use are well known in the pharmaceutical art, and are described, for example, in Remington: The Science and Practice of Pharmacy (formerly Remington's Pharmaceutical Sciences), Lippincott Williams and Wilkins (A. R. Gennaro, ed., 20^{th} Edition, 2000) and in CRC Handbook of Food, Drug, and Cosmetic Excipients, CRC Press (S. C. Smolinski, ed., 1992). For example, sterile saline and phosphate-buffered saline at physiological pH may be used. Preservatives, stabilizers, dyes and even flavoring agents may be provided in the composition. For example, benzoic acid, sodium benzoate, sorbic acid and esters of p-hydroxybenzoic acid may be added as preservatives. In addition, antioxidants and suspending agents may be used. In a preferred embodiment, an anti-microtubule agent dispersed by or in a carrier can then be added to a protein or a polysaccharide for delivery to a target site (e.g., an arthritic joint) or to a patient suffering from an inflammatory disease. Alternatively, the anti-microtubule agent can be dispersed by a carrier that forms a nanoparticle or a microemulsion, and then combined with a protein or a polysaccharide for delivery to a target or patient. In a preferred embodiment, the compositions of the instant invention are administered to a patient that is a mammal, more preferably the mammal is a human, a horse or a dog.

As noted above, certain polysaccharides and polypeptides may function as a carrier in the compositions of the instant invention. These include compositions which contain α-, β- and γ-cyclodextrin complexes that may increase the solubility of paclitaxel (e.g., Cserhati et al., J. Pharm. Biomed. Anal. 13:533-41, 1995; Grosse et al., Eur. J. Cancer 34: 68-74, 1998; Lee et al., Carbohyd. Res. 334:119-26, 2001; Sharma et al., J. Pharm. Sci. 84:1223-30, 1995; Dordunoo & Burt, Int. J. Pharm. 133:191-201, 1996) and paclitaxel complexed with albumin in drug:polymer molar ratios of between 1:1 and 4:1 (e.g., Purcell et al., Biochim. Biophys. Acta 1478:61-8, 2000); non-polymeric nanoparticles of paclitaxel which are stabilized with a coating of a protein such as albumin (WO 00/71079; WO 98/14174); conjugates of paclitaxel and amino acids including L-glutamic acid and poly(L-glutamic acid) (e.g., Li et al., Cancer Chemother Pharmacol 2000(46) 416-22); conjugates of paclitaxel and hyaluronic acid prepared using the type of chemistry described by Luo et al., Biomolecules 1:208-18, 2000). Thus, a composition comprising an anti-microtubule agent dispersed in a carrier could be made as was known in the art and described herein, which may then be suitably combined with a polypeptide or polysaccharide.

Within certain aspects of the present invention, the therapeutic composition should be biocompatible, and release one or more therapeutic agents over a period of several days to months. Further, therapeutic compositions of the present invention should preferably be stable for several months and capable of being produced or maintained or both under sterile conditions.

Within certain aspects of the present invention, therapeutic compositions may be dispersed in the form comprising any size ranging from 5 nm to 500 μm, depending upon the particular use and the dispersion form (e.g., micelle, nanoparticles, and microsphere). In certain embodiments, when the anti-microtubule agent is dispersed in a carrier that forms micelles, the micelles preferably have an average diameter in the range from about 10 nm to about 200 nm, more preferably 15 nm to about 150 nm, and most preferably 20 nm to about 100 nm. Alternatively, such compositions may also be readily applied as a spray, which can then solidify into a film, coating, or wrap on the surface to which the composition is applied. In certain embodiments, sprays may be prepared from microspheres having a wide array of sizes, which may range, for example, from 0.1 μm to 10 μm, from 10 μm to 30 μm and from 30 μm to 100 μm.

Within yet other aspects of the invention, the therapeutic compositions of the present invention may be formed as a film. Preferably, such films are generally less than 5, 4, 3, 2 or 1 mm thick, more preferably less than 0.75 mm or 0.5 mm thick, and most preferably less than 500 μm. Such films are preferably flexible with a good tensile strength (e.g., greater than 50, preferably greater than 100, and more preferably greater than 150 or 200 N/cm²), good adhesive properties (i.e., readily adheres to moist or wet surfaces), and have controlled permeability.

More preferably, therapeutic compositions of the present invention may be prepared in a variety of paste or gel forms. For example, within one embodiment of the invention, therapeutic compositions are provided which are liquid at one temperature (e.g., temperature greater than 37° C.) and solid or semisolid at another temperature (e.g., ambient body temperature, or any temperature lower than 37° C.). In a more preferable embodiment, the polypeptide or polysaccharide forms a hydrogel. Such hydrogels comprise a polypeptide or polysaccharide in aqueous solution, which will be capable of absorbing more aqueous solution if added without losing the hydrogel characteristics. For example, an aqueous solution of hyaluronic acid having a non-proinflammatory molecular mass (greater than about 900 kDa) and a concentration of about 10 mg/ml would be in the form of a hydrogel. The aqueous solution may further comprise one or more excipients that serve as a carrier for the anti-microtubule agent(s) or serve other functions, such as buffering, anti-microbial stabilization, or prevention of oxidation.

In a preferred embodiment, a carrier forms micelles in the anti-microtubule composition, wherein the micelles contain an anti-microtubule agent. Preferably, the carrier that forms micelles comprises chitosan or derivative thereof, or an amphiphilic block copolymer. In certain embodiments, the block copolymer comprises a polyester hydrophobic block and a polyether hydrophilic block copolymer, or the block copolymer comprises a hydrophilic polyether block and a hydrophobic polyether block. In yet other embodiments, the carrier that forms micelles comprises a biodegradable component. In other embodiments, the micelles have an average diameter ranging from about 10 nm to about 200 nm, more preferably an average diameter ranging from about 15 nm to about 150 nm, and most preferably an average diameter ranging from about 20 nm to about 100 nm. Instill other embodiments, the carrier forms nanoparticles containing an anti-microtubule agent, wherein the nanoparticles may further be either nanospheres or nanocapsules.

In still another embodiment, the carrier comprises a co-solvent, wherein the co-solvent is miscible with water at a concentration of at least 10% v/v in water, and the anti-microtubule agent is soluble in a mixture of water and the co-solvent. In preferred embodiments, the co-solvent is one or more of ethanol, glycerol, ethoxydiglycol, N-methylpyrrolidinone (NMP), polyethyelene glycol (PEG) or a PEG derivative with a molecular weight of up to about 750 g/mol, or dimethylsulfoxide, and more preferably is one or more of PEG 200, PEG 300, ethanol, ethoxydiglycol, and NMP. Most preferred is one or both of PEG 200 and PEG 300. In other preferred embodiments, the anti-microtubule agent is a taxane, discodermolide, colchicine, vinca alkaloids, and analogues or derivatives of any of these, more preferably the anti-microtubule agent comprises a taxane, wherein the taxane is paclitaxel or an analog or derivative thereof, and most preferably the taxane is paclitaxel. In all of the embodiments described herein, one or more carriers may likewise be utilized to disperse and deliver the therapeutic agents, such as paclitaxel or an analogue or derivative thereof.

In one embodiment, the carrier forms an oil-in-water type emulsion, the emulsion comprising a dispersed non-aqueous phase containing the anti-microtubule agent, and a continuous phase comprising water. In a preferred embodiment, the non-aqueous phase of the emulsion comprises at least one of benzyl benzoate, tributyrin, triacetin, safflower oil and corn oil. Preferably, the dispersed phase is in droplets comprising an average diameter of less than about 100 nm, more preferably less than about 200 nm, and most preferably less than about 300 nm. In one embodiment, the emulsion may be a microemulsion.

In another aspect, the carrier may take the form of a microemulsion. Emulsions and microemulsions may be prepared having a range of water content from less than 10% to greater than 70%, providing the other ingredients (a lipophilic phase and a surfactant being one or more co-surfactants) are in the correct proportions. A lipophilic phase may contain, for example, biocompatible oils or Labrafac® lipophile. Other exemplary microemulsion ingredients, including surfactants and co-surfactants, include Labrasol® (PEG 8 caprylic/capric glycerides), Gelot® 64 (Glyceryl stearate and PEG-75 Stearate), Tefose® 63 (PEG-6 Stearate and Glycol Stearate and PEG-32 Stearate), Plurol® Diisostearique (polyglyceryl-3-diisostearate, CAS 66082-42-6), Plurol® Oleique (polyglyceryl-6-dioleate, Transcutol® (ethoxydiglycol), Labrafil® (e.g., Labrafil® M 1944 CS, Oleoyl Macrogol-6 glycerides), Labrafac® PG (propylene glycol caprylate/caprate), Peceol® (glyceryl monooleate) (Gattefosse, Westwood, N.J.); and propylene glycol. An exemplary surfactant system is Labrasol®:Plurol® oleique in a ratio of 31.3:13.26. This surfactant system may be used with a lipophilic phase such as Labrafac® Lipophile in a microemulsion containing from 9 to at least 40% water.

Under certain circumstances, the compositions of the instant invention may need to be diluted, such as for use in an intravenous bag. In one embodiment, there is provided a diluted composition prepared by the process of combining a composition according to any one of claims 1-52 with an aqueous solution comprising at least one of sodium chloride, sodium phosphate salt, monosaccharide, and disaccharide. In a preferred embodiment, the anti-microtubule agent is present in the diluted composition at a concentration of about 0.01 mg/ml to about 75 mg/ml, more preferably at a concentration of about 0.1 mg/ml to about 10 mg/ml, and most preferably at a concentration of about 0.1 mg/ml to about 1.5 mg/ml.

As discussed in more detail below, anti-microtubule agents of the present invention that are optionally incorporated within one of the carriers described herein to form an effective composition, may be prepared and utilized to enhance the effects of brachytherapy by sensitizing the hyperproliferating cells that characterize the diseases being treated.

In other aspects, the compositions of the present invention are sterile. Many pharmaceuticals are manufactured to be sterile and this criterion is defined by the USP XXII <1211>. Sterilization in this embodiment may be accomplished by a number of means accepted in the industry and listed in the USP XXII <1211>, including without limitation autoclaving, dry heat, gas sterilization, ionizing radiation, and filtration. Sterilization may be maintained by what is termed aseptic processing, defined also in USP XXII <1211>. Acceptable gases used for gas sterilization include ethylene oxide. Acceptable radiation types used for ionizing radiation methods include gamma, for instance, from a cobalt 60 source and electron beam. A typical dose of gamma radiation is 2.5 MRad. Filtration may be accomplished using a filter with suitable pore size, such as 0.22 μm, and of a suitable material, such as Teflon™. In one aspect, when the polysaccharide is hyaluronic acid (HA) or a derivative thereof, the sterilization should be by a method other than irradiation as the HA tends to lose stability after exposure to γ radiation. Furthermore, a sterile composition may be achieved by using a combination of these sterilization methods and optionally aseptic techniques.

In another aspect, the compositions of the present invention are contained in a container that allows them to be used for their intended purpose, i.e., as a pharmaceutical composition. Properties of the container that are important are a volume of empty space to allow for the addition of a constitution medium, such as water or other aqueous medium (e.g., saline), an acceptable light transmission characteristic in order to prevent light energy from damaging the composition in the container (refer to USP XXII <661>), an acceptable limit of extractables within the container material (refer to USP XXII), and an acceptable barrier capacity for moisture (refer to USP XXII <671>) or oxygen. In the case of oxygen penetration, this may be controlled by including in the container a positive pressure of an inert gas such as high purity nitrogen, or a noble gas such as argon. The term “USP” refers to U.S. Pharmacopeia (see www.usp.org, Rockville, Md.).

Typical materials used to make containers for pharmaceuticals include USP Type I through III and Type NP glass (refer to USP XXII <661>), polyethylene, Teflon, silicone, and gray-butyl rubber. For parenterals, USP Types I to III glass and polyethylene are preferred. In addition, a container may contain more than one chamber (e.g., a dual chamber syringe) to allow extrusion and mixing of separate solutions to generate a single bioactive composition. In one embodiment, an anti-microtubule agent dispersed by a carrier may be in a first delivery chamber and a polypeptide or a polysaccharide may be in a second delivery chamber.

In one aspect, the compositions of the present invention include one or more preservatives or bacteriostatic agents present in an effective amount to preserve a composition and/or inhibit bacterial growth in a composition, for example, bismuth tribromophenate, methyl hydroxybenzoate, bacitracin, ethyl hydroxybenzoate, propyl hydroxybenzoate, erythromycin, chlorocresol, benzalkonium chlorides, and the like. Examples of the preservative include paraoxybenzoic acid esters, chlorobutanol, benzylalcohol, phenethyl alcohol, dehydroacetic acid, sorbic acid, etc. In one aspect, the compositions of the present invention include one or more bactericidal (also known as bacteriacidal) agents. In one aspect, the compositions of the present invention include one or more antioxidants, present in an effective amount. Examples of the antioxidant include sulfites and ascorbic acid. In one aspect, the compositions of the present invention include one or more coloring agents, also referred to as dyestuffs, which will be present in an effective amount to impart observable coloration to the composition. Examples of coloring agents include dyes suitable for food such as those known as F. D. & C. dyes, and natural coloring agents such as grape skin extract, beet red powder, beta carotene, annato, carmine, turmeric, paprika, and so forth.

In certain embodiments, the compositions of the present invention are subjected to a process of lyophilization, comprising lyophilization of any of the compositions described above to create a lyophilized powder. In addition, the compositions of the invention may be spray dried as described above. In a preferred embodiment, the process further comprises reconstitution of the lyophilized powder with water or other aqueous media, such as benzyl alcohol-containing bacteriostatic water for injection, to create a reconstituted solution (Bacteriostatic Water for Injection, Abbott Laboratories, Abbott Park, Ill.).

The compositions may be administered to a patient as a single dosage unit or form (e.g., film or gel), and the compositions may be administered as a plurality of dosage units (e.g., in aerosol form as a spray). For example, the anti-microtubule agent formulations may be sterilized and packaged in single-use, plastic laminated pouches or plastic tubes of dimensions selected to provide for routine, measured dispensing. In one example, the container may have dimensions anticipated to dispense 0.5 ml of the anti-microtubule agent composition (e.g., a gel form) to a limited area of a target site or in a subject to treat or prevent an inflammatory condition. A typical target, for example, is in the immediate vicinity of or within an arthritic joint. In another aspect, the compositions of the instant invention may also be formulated for use in vitro, such as in experimental systems in the laboratory.

Also provided is a process for making the compositions of the instant invention. In one embodiment, a process for forming a composition comprises (a) contacting an anti-microtubule agent with a carrier to form an anti-microtubule agent dispersed by a carrier, and (b) combining (a) with a polypeptide or a polysaccharide, thereby forming the composition. In another embodiment, a process for forming a composition comprises (a) combining a polypeptide or a polysaccharide with a carrier in an aqueous medium, and (b) adding an anti-microtubule agent to (a), thereby forming a composition wherein the anti-microtubule agent is dispersed by the carrier. In one embodiment, the polypeptide or polysaccharide is a polysaccharide as described herein and in anther embodiment the polypeptide or polysaccharide is a polypeptide as described herein. In a preferred embodiment, the process for forming a composition results in a carrier that forms micelles, the micelles containing an anti-microtubule agent. Preferably, the carrier that forms micelles comprises chitosan or derivative thereof, or an amphiphilic block copolymer. In certain embodiments, the block copolymer comprises a polyester hydrophobic block and a polyether hydrophilic block copolymer, or the block copolymer comprises a hydrophilic polyether block and a hydrophobic polyether block. In yet other embodiments, the carrier that forms micelles comprises a biodegradable component. In other embodiments, the micelles have an average diameter ranging from about 10 nm to about 200 nm, more preferably an average diameter ranging from about 15 nm to about 150 nm, and most preferably an average diameter ranging from about 20 nm to about 100 nm.

In still other embodiments, the process will include producing a composition with a carrier that forms nanoparticles containing an anti-microtubule agent, wherein the nanoparticles may further be, without limitation, nanospheres, nanocapsules, nanocrystals, and the like. Nanoparticles may comprise a polymeric material forming either a continuous matrix within the nanoparticle, with the anti-microtubule agent contained therein, or may form a capsule surrounding a region of the nanoparticle in which the anti-microtubule agent is contained, or in which the majority of the anti-microtubule agent is contained. Suitable polymeric materials may comprise one or more of polyesters (e.g., polylactide, polyglycolide, polycaprolactone, polybutyrolactone, polyvalerolactone and copolymers containing repeating units of at least two of these homopolymers), polyorthoesters, polyanhydrides, polythioesters, polyamides, methacrylates, polyethylene glycols, polyethylene glycol-propylene glycol copolymers, polyamino acids, cellulose and cellulose derivatives previously listed, chitosan, and copolymers of any of these listed polymers, including without limitation alternating, block, graft and random copolymers. In still another embodiment, the carrier comprises a co-solvent, wherein the co-solvent is miscible with water at a concentration of at least 10% v/v in water, and the anti-microtubule agent is soluble in a mixture of water and the co-solvent. In preferred embodiments, the co-solvent is one or more of ethanol, glycerol, ethoxydiglycol, N-methylpyrrolidinone (NMP), polyethyelene glycol (PEG) or a PEG derivative with a molecular weight of up to about 750 g/mol, or dimethylsulfoxide, and more preferably is one or more of PEG 200, PEG 300, ethanol, ethoxydiglycol, and NMP. In other preferred embodiments, the anti-microtubule agent is a taxane, discodermolide, colchicine, vinca alkaloids, and analogues or derivatives of any of these, more preferably the anti-microtubule agent comprises a taxane, wherein the taxane is paclitaxel or an analog or derivative thereof, and most preferably the taxane is paclitaxel. In certain preferred embodiments, the process will yield composition in a form selected from a gel, a hydrogel, a film, a paste, a cream, a spray, an ointment, a paste, or a wrap, more preferably a hydrogel.

In other preferred processes, the polypeptide or polysaccharide is suspended or dissolved in an aqueous medium prior to combination with the dispersed anti-microtubule agent, which may be useful for forming a composition with the desired consistency, such as a gel or hydrogel. Preferably, the process of making a composition according to the instant invention is further sterilized by at least one of autoclaving, radiation, or filtering. In other embodiments, the compositions formed by the processes described herein are further lyophilized or spray dried. In addition, there is contemplated by the instant invention a composition produced by any of the aforementioned processes.

The present invention also contemplates kits for making a composition to treat an inflammatory condition. Such kits comprise one or more containers. In one aspect, the kit comprises an anti-microtubule agent dispersed by a carrier and a polysaccharide or a polypeptide. In a preferred embodiment, the kit comprises first container having an anti-microtubule agent dispersed by a carrier and a second container having a polysaccharide or a polypeptide. In a further preferred embodiment, the anti-microtubule agent dispersed by a carrier is in a form selected from the group consisting of a micelle, a nanoparticle, a microsphere, a liposome, an emulsion, a microemulsion, a cyclodextrin-complex, a co-solvent media, and a surfactant containing media, and most preferably a micelle. In another preferred embodiment, the polysaccharide or polypeptide is in the form of a solid, a liquid, a gel, or a hydrogel, and most preferably a hydrogel. In one aspect, the polypeptide or polysaccharide is a polypeptide selected from a polyamino acid homopolymer, a polyamino acid copolymer, a collagen, an albumin, a fibrin, a gelatin, and derivatives thereof. In another aspect, the polypeptide or polysaccharide is a polysaccharide selected from hyaluronic acid, hyaluronic acid derivatives, cellulose, cellulose derivatives, chitosan, chitosan derivatives, dextran, and dextran derivatives, and most preferably is hyaluronic acid or a derivative thereof. In a more preferred embodiment, the anti-microtubule agent is paclitaxel or an analogue or derivative thereof, and most preferably is paclitaxel. In other aspects, the anti-microtubule agent is dispersed in an aqueous medium or at least one of the kit components is lyophilized or spray dried.

A kit will also comprise written material describing the use of an anti-microtubule agent composition of the present invention for treating an inflammatory disease or target site. In one preferred embodiment, the written material will provide that the polysaccharide or polypeptide is suspended or dissolved in an aqueous medium prior to combination with the dispersed anti-microtubule agent. The written material can be applied directly to a container or the written material can be provided in the form of a packaging insert.

III. Clinical Applications

In order to further the understanding of the compositions and methods for the treatment of inflammatory conditions, representative clinical applications are discussed in more detail below. As utilized herein, it should be understood that the term “treatment” refers to the therapeutic administration of a desired composition or compound in an amount and/or for a time sufficient to treat, inhibit, or prevent at least one aspect or marker of an inflammatory disease, in a statistically or clinically significant manner. The therapeutic efficacy of an anti-microtubule agent composition according to the present invention is based on a successful clinical outcome and does not require 100% elimination of the symptoms associated with an inflammatory disease. For example, achieving a level of anti-microtubule agent activity at the site of inflammation, which allows the patient to resolve or otherwise eradicate the inflammation symptoms, or allows the patient to have a better quality of life, is sufficient.

In certain preferred embodiments, there is provided by the instant invention a method for treating an inflammatory condition, comprising administering to a patient in need thereof a therapeutically effective amount of a composition comprising an anti-microtubule agent composition as described herein. In another embodiment, the method comprises delivering an anti-microtubule agent to a target site, wherein the method comprises forming an anti-microtubule agent composition as described herein, and introducing the anti-microtubule agent composition into an aqueous environment, wherein a target site is in contact with the aqueous environment. Preferably, an inflammatory condition treated with the above methods may be inflammatory arthritis, adhesions, tumor excision sites, fibroproliferative ocular conditions, and the like. In certain embodiments, the composition used in the above methods is in a form selected from the group consisting of a gel, a hydrogel, a film, a paste, a cream, a spray, an ointment, or a wrap. Preferably, the above methods are used to administer the compositions described herein by a route selected from intraarticular, intraperitoneal, topical, intravenous, ocular, or to the resection margin of tumors. In more preferred embodiments, the anti-microtubule agent used in the compositions of these methods is paclitaxel or an analog or derivative thereof, and most preferably is paclitaxel. In preferred embodiments, the above methods are used to administer the anti-microtubule compositions described herein to a patient in need thereof who is a mammal, and more preferably the mammal is a human, horse, or dog.

A. Inflammatory Arthritis

As noted above, methods are provided for treating or preventing inflammatory arthritis (e.g., osteoarthritis or rheumatoid arthritis) comprising the step of administering to a patient a therapeutically effective amount of an anti-microtubule agent. Inflammatory arthritis includes a variety of conditions including, but not limited to, rheumatoid arthritis, systemic lupus erythematosus, systemic sclerosis (scleroderma), mixed connective tissue disease, Sjögren's syndrome, ankylosing spondylitis, Behçet's syndrome, sarcoidosis, and osteoarthritis—all of which feature inflamed, painful joints as a prominent symptom. Within a preferred embodiment of the invention, anti-microtubule agents may be administered directly to a joint by intra-articular injection, as a surgical paste, or administered by another route, e.g., systemically or orally.

Suitable anti-microtubule agents are discussed in detail above, and include, for example, taxanes, discodermolide, colchicine and CI 980 (Allen et al., Am. J. Physiol. 261(4 Pt. 1): L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), vinca alkaloids (e.g., vinblastine and vincristine) (ter Haar et al., Biochemistry 35: 243-250, 1996), as well as analogues and derivatives of any of these.

Such agents may, within certain embodiments, be delivered as a composition along with a polymeric carrier, or in a liposome formulation as discussed in more detail both above and below.

An effective anti-microtubule therapy for inflammatory arthritis will accomplish one or more of the following: (i) decrease the severity of symptoms (pain, swelling and tenderness of affected joints; morning stiffness, weakness, fatigue, anorexia, weight loss); (ii) decrease the severity of clinical signs of the disease (thickening of the joint capsule, synovial hypertrophy, joint effusion, soft tissue contractures, decreased range of motion, ankylosis and fixed joint deformity); (iii) decrease the extra-articular manifestations of the disease (rheumatic nodules, vasculitis, pulmonary nodules, interstitial fibrosis, pericarditis, episcleritis, iritis, Felty's syndrome, osteoporosis); (iv) increase the frequency and duration of disease remission/symptom-free periods; (v) prevent fixed impairment and disability; and/or (vi) prevent/attenuate chronic progression of the disease. Pathologically, an effective anti-microtubule therapy for inflammatory arthritis will produce at least one of the following: (i) decrease the inflammatory response, (ii) disrupt the activity of inflammatory cytokines (such as IL-1, TNF□, FGF, VEGF), (iii) inhibit synoviocyte proliferation, (iv) block matrix metalloproteinase activity, and/or (v) inhibit angiogenesis. An anti-microtubule agent will be administered via intra-articular injection in the minimum dose to achieve any or all of the above-mentioned results. The polypeptide or polysaccharide may itself confer biological activity to the composition in the sense that if given alone, the polypeptide or polysaccharide may provide some therapeutic benefit. In one aspect, an anti-microtubule agent may provide a similar, different, or additional therapeutic benefit according to the described classification. In another aspect, the present invention contemplates complimentary, additive, or synergistic therapeutic effects. Complimentary effects may be assessed independently, whereas synergistic effects may be assessed by a single set of criteria.

In certain aspects, an anti-microtubule agent can be administered in any manner sufficient within any of the compositions described herein to achieve the above endpoints. However, the preferred method of administration is intra-articular injection of the anti-microtubule drug with a protein or polysaccharide in a carrier selected from a co-solvent system, a micellar, liposomal or nanoparticulate dispersion. In one embodiment, the polysaccharide is preferably hyaluronic acid or its sodium salt or a hydrogel comprising one of these, and an Anti-microtubule agent, paclitaxel and its carrier are contained therein.

The anti-microtubule agent can be administered as a chronic low dose therapy (e.g., at least three repeated weekly or monthly intra-articular injections) to prevent disease progression, prolong disease remission, or decrease symptoms in active disease. Alternatively, the therapeutic agent can be administered in higher doses as a “pulse” therapy (e.g., 1-3 intra-articular injections of higher dose therapy administered weekly to monthly) to induce remission in acutely active disease. The minimum dose capable of achieving these endpoints can be used and can vary according to patient, severity of disease and formulation of the administered agent.

In certain preferred embodiments, for example, the following anti-microtubule agents and dosing schedules could be given every 4 to 12 weeks to a patient in need thereof, as tolerated, in a carrier (such as a micelle) combined with a polysaccharide (such as hyaluronic acid or a derivative thereof). Preferably, these compositions are administered by intra-articular injection.

| Anti-microtubule agent | Dose Range | |
|---|---|---|
| Paclitaxel | 1-10 | mg |
| Docetaxol | 0.5-10 | mg |
| Vincristine Sulfate | 0.01-2 | mg |
| Vinblastine Sulfate | 0.2-1 | mg |
| Colchicine | 1-10 | mg |

In certain other embodiments where an inflammatory disease is more aggressive, a preferred method of administration of the exemplary anti-microtubule agents could be given every 1 to 4 weeks for a total of 1 to 6 doses, as tolerated, or until symptoms subside, as follows:

| Anti-microtubule agent | Dose Range | |
|---|---|---|
| Paclitaxel | 10-75 | mg |
| Docetaxol | 5-25 | mg |
| Vincristine Sulfate | 0.2-1 | mg |
| Vinblastine Sulfate | 0.4-4 | mg |
| Colchicine | 4-5 | mg |

Thus, one preferred embodiment is a composition comprising an anti-microtubule agent dispersed by a carrier and hyaluronic acid or a derivative thereof, the composition being in sterile form. Preferably, the anti-microtubule agent dispersed by a carrier is in the form of a micelle, a nanosphere, or a co-solvent composition. Most preferably, the anti-microtubule agent is paclitaxel or a derivative thereof, more preferably is paclitaxel, and most preferably is dispersed in the form of a hydrogel.

B. Adhesions

Adhesion formation, a complex process in which bodily tissues that are normally separate grow together, is most commonly seen to occur as a result of surgical trauma. These post-operative adhesions occur in 60 to 90% of patients undergoing major gynacologic surgery and represent one of the most common causes of intestinal obstruction and infertility in the industrialized world. Other adhesion-treated complications include chronic pelvic pain, urethral obstruction and voiding dysfunction. Currently, preventative therapies, such inert surgical barriers made of hyaluronic acid or cellulose placed at the operative site at the time of surgery, are used to inhibit adhesion formation. Various modes of adhesion prevention have been examined, including (1) prevention of fibrin deposition, (2) reduction of local tissue inflammation and (3) removal of fibrin deposits. Fibrin deposition is prevented through the use of physical barriers that are either mechanical or comprised of viscous solutions. Although many investigators are utilizing adhesion prevention barriers, a number of technical difficulties exist. Inflammation is reduced by the administration of drugs such as corticosteroids and nonsteroidal anti-inflammatory drugs. However, the results from the use of these drugs in animal models have not been encouraging due to the extent of the inflammatory response and dose restriction due to systemic side effects. Finally, the removal of fibrin deposits has been investigated using proteolytic and fibrinolytic enzymes. A potential complication to the clinical use of these enzymes is the possibility for excessive bleeding.

Thus, within other aspects of the invention, methods are provided for treating and/or preventing adhesions by administering to the patient a protein or polysaccharide containing a solubilized (e.g., micelle or liposome containing) anti-microtubule agent.

A wide variety of animal models may be utilized in order to assess a particular therapeutic composition or treatment regimen. Briefly, peritoneal adhesions occur in animals as a result of severe inflicted damage, which usually involves two adjacent surfaces. Injuries may be mechanical, due to ischemia, or due to the introduction of foreign material. Mechanical injuries include crushing of the bowel (Choate et al., Arch. Surg. 88:249-254, 1964) and stripping or scrubbing away the outer layers of bowel wall (Gustavsson et al., Acta Chir. Scand. 109:327-333, 1955). Dividing major vessels to loops of the intestine induces ischemia (James et al., J. Path. Bact. 90:279-287, 1965). Foreign material that may be introduced into the area includes talcum (Green et al., Proc. Soc. Exp. Biol. Med. 133:544-550, 1970), gauze sponges (Lehman and Boys, Ann. Surg 111:427-435, 1940), toxic chemicals (Chancy, Arch. Surg. 60:1151-1153, 1950), bacteria (Moin et al., Am. J. Med. Sci. 250:675-679, 1965) and feces (Jackson, Surgery 44:507-518, 1958).

Presently, typical adhesion prevention models include the rabbit uterine horn model, which involves the abrasion of the rabbit uterus (Linsky et al., J. Reprod. Med. 32(1):17-20, 1987), the rabbit uterine horn; devascularization modification model, which involves abrasion and devascularization of the uterus (Wiseman et al., J. Invest Surg. 7:527-532, 1994); and the rabbit cecal sidewall model which involves the excision of a patch of parietal peritoneum plus the abrasion of the cecum (Wiseman and Johns, Fertil. Steril. Suppl: 25S, 1993).

Representative anti-microtubule agents for treating adhesions are discussed in detail above, and include taxanes, colchicine and CI 980 (Allen et al., Am. J. Physiol. 261(4 Pt. 1): L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), vinca alkaloids (e.g., vinblastine and vincrystine), discodermolide (ter Haar et al., Biochemistry 35: 243-250, 1996), as well as analogues and derivatives of any of these

Utilizing the agents, compositions and methods provided herein a wide variety of adhesions and complications of surgery can be treated or prevented. Adhesion formation or unwanted scar tissue accumulation and/or encapsulation complicates a variety of surgical procedures. As described above, surgical adhesions complicate virtually any open or endoscopic surgical procedure in the abdominal or pelvic cavity. Encapsulation of surgical implants also complicates breast reconstruction surgery, joint replacement surgery, hernia repair surgery, artificial vascular graft surgery, and neurosurgery. In each case, the implant becomes encapsulated by a fibrous connective tissue capsule that compromises or impairs the function of the surgical implant (e.g., breast implant, artificial joint, surgical mesh, vascular graft, dural patch). Chronic inflammation and scarring also occurs during surgery to correct chronic sinusitis or removal of other regions of chronic inflammation (e.g., foreign bodies; infections such as fungal and mycobacterial).

The anti-microtubule agent can be administered in any manner that achieves a statistically significant result. Preferred methods include peritubular administration (either direct application at the time of surgery or with endoscopic, ultrasound, CT, MRI, or fluoroscopic guidance); “coating” the surgical implant; and placement of a drug-eluting polymeric implant at the surgical site.

For paclitaxel, a variety of embodiments are described for the management of adhesions. In one preferred embodiment, 1-15 mg of paclitaxel is loaded into a micellar carrier, combined with hyaluronic acid, and applied to the mesenteric surface as a “paste”, “film”, or “wrap,” which releases the drug over a period of time such that the incidence of surgical adhesions is reduced. During endoscopic procedures, 1-15 mg of paclitaxel contained in the combined micellar—hyaluronic acid preparation is applied as a “spray” via delivery ports in an endoscope to the mesentery of the abdominal and pelvic organs manipulated during the operation. In another preferred embodiment, 1-15 mg of paclitaxel is applied to the surface of the surgical implant (e.g., breast implant, artificial joint, vascular graft) via the micellar-hyaluronic acid composition to prevent encapsulation/inappropriate scarring in the vicinity of the implant. In yet another preferred embodiment, a micellar-hyaluronic acid implant containing 0.25-15 mg paclitaxel is applied directly to the surgical site (e.g., directly into the sinus cavity, chest cavity, abdominal cavity, or at the operative site during neurosurgery) such that recurrence of inflammation, adhesion formation, or scarring is reduced. In another embodiment, an intraperitoneal surgical lavage fluid containing 1-15 mg paclitaxel (and up to 250 mg paclitaxel if used as part of a tumor resection surgery) would be administered by a physician at the time of, or immediately following, surgery. Preferably, the lavage fluid would have the property of mucoadherence (i.e., adheres selectively to the mesenteric and peritoneal surfaces of the abdomen).

For docetaxel, a variety of embodiments are described for the management of adhesions. In a preferred embodiment, 0.5-10 mg of docetaxel is loaded into a micellar carrier, incorporated into hyaluronic acid and applied to the mesenteric surface as a “paste”, “film”, or “wrap” which releases the drug over a period of time such that the incidence of surgical adhesions is reduced. During endoscopic procedures, 0.5-10 mg of docetaxel contained in the micellar-hyaluronic acid preparation is applied as a “spray”, via delivery ports in an endoscope, to the mesentery of the abdominal and pelvic organs manipulated during the operation. In another preferred embodiment, 0.5-10 mg of docetaxel is applied to the surface of the surgical implant (e.g., breast implant, artificial joint, vascular graft) via the micellar-hyaluronic acid carrier to prevent encapsulation/inappropriate scarring in the vicinity of the implant. In yet another preferred embodiment, a micellar-hyaluronic acid implant containing 0.1-15 mg docetaxel is applied directly to the surgical site (e.g., directly into the sinus cavity, chest cavity, abdominal cavity, or at the operative site during neurosurgery) such that recurrence of inflammation, adhesion formation, or scarring is reduced. In another embodiment, an intraperitoneal surgical lavage fluid containing 0.5 to 10 mg (up to 100 mg if used as part of a tumor resection surgery) docetaxel, would be administered at the time of, or immediately following, surgery, by a physician. For this last embodiment, a fluid which has the added property of mucoadherence (i.e., adheres selectively to the mesenteric and peritoneal surfaces of the abdomen) would be preferred.

For vincristine, a variety of embodiments are described for the management of adhesions. In a preferred embodiment, 0.01-0.2 mg of vincristine sulfate is loaded into a micellar carrier, incorporated into hyaluronic acid and applied to the mesenteric surface as a “paste”, “film”, or “wrap” which releases the drug over a period of time such that the incidence of surgical adhesions is reduced. During endoscopic procedures, 0.01-0.2 mg of vincristine sulfate contained in the micellar-hyaluronic acid preparation is applied as a “spray”, via delivery ports in an endoscope, to the mesentery of the abdominal and pelvic organs manipulated during the operation. In another preferred embodiment, 0.01-0.2 mg of vincristine sulfate is applied to the surface of the surgical implant (e.g., breast implant, artificial joint, vascular graft) via the micellar-hyaluronic acid carrier to prevent encapsulation/inappropriate scarring in the vicinity of the implant. In yet another preferred embodiment, a micellar-hyaluronic acid implant containing 0.01-0.25 mg vincristine sulfate is applied directly to the surgical site (e.g., directly into the sinus cavity, chest cavity, abdominal cavity, or at the operative site during neurosurgery) such that recurrence of inflammation, adhesion formation, or scarring is reduced. In another embodiment, an intraperitoneal surgical lavage fluid containing 0.01 to 0.2 mg (up to 1.5 mg if used as part of a tumor resection surgery) vincristine sulfate, would be administered at the time of, or immediately following, surgery, by a physician. For this last embodiment, a fluid that has the added property of mucoadherence (i.e., adheres selectively to the mesenteric and peritoneal surfaces of the abdomen) would be preferred.

For vinblastine, a variety of embodiments are described for the management of adhesions. In a preferred embodiment, 0.2-1.0 mg of vinblastine sulfate is loaded into a micellar carrier, incorporated into hyaluronic acid and applied to the mesenteric surface as a “paste”, “film”, or “wrap” which releases the drug over a period of time such that the incidence of surgical adhesions is reduced. During endoscopic procedures, 0.2-1.0 mg of vinblastine sulfate contained in the micellar-hyaluronic acid preparation is applied as a “spray”, via delivery ports in an endoscope, to the mesentery of the abdominal and pelvic organs manipulated during the operation. In another preferred embodiment, 0.2-1.0 mg of vinblastine sulfate is applied to the surface of the surgical implant (e.g., breast implant, artificial joint, vascular graft) via the micellar-hyaluronic acid carrier to prevent encapsulation/inappropriate scarring in the vicinity of the implant. In yet another preferred embodiment, a micellar-hyaluronic acid implant containing 0.2 to 1.0 mg vinblastine sulfate is applied directly to the surgical site (e.g., directly into the sinus cavity, chest cavity, abdominal cavity, or at the operative site during neurosurgery) such that recurrence of inflammation, adhesion formation, or scarring is reduced. In another embodiment, an intraperitoneal surgical lavage fluid containing 0.2 to 1.0 mg (up to 3.7 mg if used as part of a tumor resection surgery) vinblastine sulfate, would be administered at the time of, or immediately following, surgery, by a physician. For this last embodiment, a fluid that has the added property of mucoadherence (i.e., adheres selectively to the mesenteric and peritoneal surfaces of the abdomen) would be preferred.

For colchicine, a variety of embodiments are described for the management of adhesions. In a preferred embodiment, 1.0-10 mg of colchicine is loaded into a micellar carrier, incorporated into hyaluronic acid and applied to the mesenteric surface as a “paste”, “film”, or “wrap” which releases the drug over a period of time such that the incidence of surgical adhesions is reduced. During endoscopic procedures, 1.0-10 mg of colchicine contained in the micellar-hyaluronic acid preparation is applied as a “spray”, via delivery ports in an endoscope, to the mesentery of the abdominal and pelvic organs manipulated during the operation. In another preferred embodiment, 1.0-10 mg of colchicine is applied to the surface of the surgical implant (e.g., breast implant, artificial joint, vascular graft) via the micellar-hyaluronic acid carrier to prevent encapsulation/inappropriate scarring in the vicinity of the implant. In yet another preferred embodiment, a micellar-hyaluronic acid implant containing 1.0-10 mg colchicine is applied directly to the surgical site (e.g., directly into the sinus cavity, chest cavity, abdominal cavity, or at the operative site during neurosurgery) such that recurrence of inflammation, adhesion formation, or scarring is reduced. In another embodiment, an intraperitoneal surgical lavage fluid containing 1.0 to 10 mg (up to 100 mg if used as part of a tumor resection surgery) cholchicine, would be administered at the time of, or immediately following, surgery, by a physician. For this last embodiment, a fluid that has the added property of mucoadherence (i.e., adheres selectively to the mesenteric and peritoneal surfaces of the abdomen) would be preferred.

C. Tumor Excision Sites

Within further aspects of the present invention, methods are provided for treating tumor excision sites, comprising administering to a patient a protein or polysaccharide containing solubilized (e.g., liposome or micelle containing) anti-microtubule agent, such that the local recurrence of cancer is inhibited.

Local recurrence of malignancy following primary surgical excision of the mass remains a significant clinical problem. In one series of breast cancer patients who underwent lumpectomy of a primary breast tumor, almost ⅔ of the patients that presented with recurrent disease had local (i.e., tumor in the same breast) disease, while only ⅓ presented with metastatic disease. Other pathological studies have demonstrated that most local tumor recurrence occurs within a 2 cm margin of the primary resection margin. Therefore, treatments designed to address this problem are greatly needed. Local recurrence is also a significant problem in the surgical management of brain tumors. For example, within one embodiment of the invention, anti-microtubule compositions may be administered to the site of a neurological tumor subsequent to excision, such that recurrence of the brain tumor (benign or malignant) is inhibited. Briefly, the brain is highly functionally localized; i.e., each specific anatomical region is specialized to carry out a specific function. Therefore it is the location of brain tumor pathology that is often more important than the type. A relatively small lesion in a key area can be far more devastating than a much larger lesion in a less important area. Similarly, a lesion on the surface of the brain may be easy to resect surgically, while the same tumor located deep in the brain may not (one would have to cut through too many vital structures to reach it). Also, even benign tumors can be dangerous for several reasons: they may grow in a key area and cause significant damage; even though they would be cured by surgical resection this may not be possible; and finally, if left unchecked they can cause increased intracranial pressure. The skull is an enclosed space incapable of expansion. Therefore, if something is growing in one location, something else must be being compressed in another location—the result is increased pressure in the skull or increased intracranial pressure. If such a condition is left untreated, vital structures can be compressed, resulting in death. The incidence of CNS (central nervous system) malignancies is 8-16 per 100,000. The prognosis of primary malignancy of the brain is dismal, with a median survival of less than one year, even following surgical resection. These tumors, especially gliomas, are predominantly a local disease that recurs within 2 centimeters of the original focus of disease after surgical removal.

Representative examples of brain tumors which may be treated utilizing the compositions and methods described herein include Glial Tumors (such as Anaplastic Astrocytoma, Glioblastoma Multiform, Pilocytic Astrocytoma, Oligodendroglioma, Ependymoma, Myxopapillary Ependymoma, Subependymoma, Choroid Plexus Papilloma); Neuron Tumors (e.g., Neuroblastoma, Ganglioneuroblastoma, Ganglioneuroma, and Medulloblastoma); Pineal Gland Tumors (e.g., Pineoblastoma and Pineocytoma); Menigeal Tumors (e.g., Meningioma, Meningeal Hemangiopericytoma, Meningeal Sarcoma); Tumors of Nerve Sheath Cells (e.g., Schwannoma (Neurolemmoma) and Neurofibroma); Lymphomas (e.g., Hodgkin's and Non-Hodgkin's Lymphoma (including numerous subtypes, both primary and secondary); Malformative Tumors (e.g., Craniopharyngioma, Epidermoid Cysts, Dermoid Cysts and Colloid Cysts); and Metastatic Tumors (which can be derived from virtually any tumor, the most common being from lung, breast, melanoma, kidney, and gastrointestinal tract tumors).

As noted above, representative anti-microtubule agents for treating adhesions are discussed in detail above, and include taxanes, colchicine and CI-980 (Allen et al., Am. J. Physiol. 261(4 Pt. 1): L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), vinca alkaloids (e.g., vinblastine and vincristine), discodermolide (ter Haar et al., Biochemistry 35: 243-250, 1996), as well as analogues and derivatives of any of these

Within one embodiment of the invention, the compound or composition is administered directly to the tumor excision site (e.g., applied by swabbing, brushing or otherwise coating the resection margins of the tumor with the antimicrotubule composition(s)). Within particularly preferred embodiments of the invention, the antimicotubule compositions are applied after hepatic resections for malignancy, colon tumor resection surgery, breast tumor lumpectomy and after neurosurgical tumor resection operations.

For paclitaxel, a variety of embodiments are described for the management of local tumor recurrence. In one preferred embodiment, 1-25 mg of paclitaxel is loaded into a micellar carrier, incorporated into hyaluronic acid and applied to the resection surface as a “paste”, “film”, or “gel” which releases the drug over a period of time such that the incidence of tumor recurrence is reduced. During endoscopic procedures, 1-25 mg of paclitaxel contained in the micellar-hyaluronic acid preparation is applied as a “spray”, via delivery ports in an endoscope, to the resection site. In another embodiment, an intraperitoneal surgical lavage fluid containing 10 to 250 mg paclitaxel is administered at the time of, or immediately following, surgery. For this last embodiment, a fluid that has the added property of mucoadherence (i.e., adheres selectively to the mesenteric and peritoneal surfaces of the abdomen) would be preferred.

For docetaxel, a variety of embodiments are described for the management of local tumor recurrence. In one preferred embodiment, 0.5-15 mg of docetaxel is loaded into a micellar carrier, incorporated into hyaluronic acid and applied to the resection surface as a “paste”, “film”, or “gel” which releases the drug over a period of time such that the incidence of tumor recurrence is reduced. During endoscopic procedures, 0.5-15 mg of docetaxel contained in the micellar-hyaluronic acid preparation is applied as a “spray”, via delivery ports in an endoscope, to the resection site. In another embodiment, an intraperitoneal surgical lavage fluid containing 10 to 100 mg docetaxel is administered at the time of, or immediately following, surgery. For this last embodiment, a fluid which has the added property of mucoadherence (i.e., adheres selectively to the mesenteric and peritoneal surfaces of the abdomen) would be preferred.

For vincristine sulfate, a variety of embodiments are described for the management of local tumor recurrence. In one preferred embodiment, 0.05-1.0 mg of vincristine sulfate is loaded into a micellar carrier, incorporated into hyaluronic acid and applied to the resection surface as a “paste”, “film”, or “gel” which releases the drug over a period of time such that the incidence of tumor recurrence is reduced. During endoscopic procedures, 0.05-1.0 mg of vincristine sulfate contained in the micellar-hyaluronic acid preparation is applied as a “spray”, via delivery ports in an endoscope, to the resection site. In another embodiment, an intraperitoneal surgical lavage fluid containing 0.1 to 2.0 mg vincristine sulfate is administered at the time of, or immediately following, surgery. For this last embodiment, a fluid that has the added property of mucoadherence (i.e., adheres selectively to the mesenteric and peritoneal surfaces of the abdomen) would be preferred. For vinblastine sulfate, a variety of embodiments are described for the management of local tumor recurrence. In one preferred embodiment, 0.1-2.0 mg of vinblastine sulfate is loaded into a micellar carrier, incorporated into hyaluronic acid and applied to the resection surface as a “paste”, “film”, or “gel” which releases the drug over a period of time such that the incidence of tumor recurrence is reduced. During endoscopic procedures, 0.1-2.0 mg of vinblastine sulfate contained in the micellar-hyaluronic acid preparation is applied as a “spray”, via delivery ports in an endoscope, to the resection site. In another embodiment, an intraperitoneal surgical lavage fluid containing 1.0 to 15 mg vinblasitne sulfate is administered at the time of, or immediately following, surgery. For this last embodiment, a fluid that has the added property of mucoadherence (i.e., adheres selectively to the mesenteric and peritoneal surfaces of the abdomen) would be preferred.

For cholchicine, a variety of embodiments are described for the management of local tumor recurrence. In one preferred embodiment, 0.5-4.0 mg of cholchicine is loaded into a micellar carrier, incorporated into hyaluronic acid and applied to the resection surface as a “paste”, “film”, or “gel” which releases the drug over a period of time such that the incidence of tumor recurrence is reduced. During endoscopic procedures, 0.5-4.0 mg of cholchicine contained in the micellar-hyaluronic acid preparation is applied as a “spray”, via delivery ports in an endoscope, to the resection site. In another embodiment, an intraperitoneal surgical lavage fluid containing 10 to 100 mg cholchicine is administered at the time of, or immediately following, surgery. For this last embodiment, a fluid which has the added property of mucoadherence (i.e., adheres selectively to the mesenteric and peritoneal surfaces of the abdomen) would be preferred.

D. Fibroproliferative Ocular Conditions

As noted above, the present invention also provides methods for treating fibroproliferative ocular conditions, including for example, corneal neovascularization, neovascular glaucoma, proliferative diabetic retinopathy, retrolental fibroblasia, macular degeneration, posterior lens opacification following cataract surgery and failure of glaucoma filtration surgery due to scarring.

Briefly, corneal neovascularization as a result of injury to the anterior segment is a significant cause of decreased visual acuity and blindness, and a major risk factor for rejection of corneal allografts. Currently no clinically satisfactory therapy exists for inhibition of corneal neovascularization or regression of existing corneal new vessels. Topical corticosteroids appear to have some clinical utility, presumably by limiting stromal inflammation.

Thus, within one aspect of the present invention methods are provided for treating fibroproliferative diseases of the eye such as corneal neovascularization (including corneal graft neovascularization), comprising the step of administering to a patient a therapeutically effective amount of an antimicrotubule composition (as described above) to the cornea, such that the formation of blood vessels is inhibited. Briefly, the cornea is a tissue which normally lacks blood vessels. In certain pathological conditions however, capillaries may extend into the cornea from the pericorneal vascular plexus of the limbus. When the cornea becomes vascularized, it also becomes clouded, resulting in a decline in the patient's visual acuity. Visual loss may become complete if the cornea completely opacitates.

A wide variety of disorders can result in corneal neovascularization, including for example, corneal infections (e.g., trachoma, herpes simplex keratitis, leishmaniasis and onchocerciasis), immunological processes (e.g., graft rejection and Stevens-Johnson's syndrome), alkali burns, trauma, inflammation (of any cause), toxic and nutritional deficiency states, and as a complication of wearing contact lenses.

Within particularly preferred embodiments of the invention, the compositions provided herein can be prepared for topical administration in saline (combined with any of the preservatives and antimicrobial agents commonly used in ocular preparations), and administered in eyedrop form. Topical therapy may also be useful prophylactically in corneal lesions, which are known to have a high probability of inducing an fibroproliferative response (such as chemical burns). In these instances the treatment, likely in combination with steroids, may be instituted immediately to help prevent subsequent complications.

Within other embodiments, the compositions described above may be injected directly into the eye by an ophthalmologist under microscopic guidance. The preferred site of injection may vary with the morphology of the individual lesion, but the goal of the administration would be to place the composition in a region at risk for the development of fibroproliferative scar tissue (i.e., interspersed between the blood vessels and the normal cornea in corneal neovascularization, around/coated on a surgically implanted lens in cataract surgery, in or around a surgically created drainage site in glaucoma filtration surgery, into the vitreous/around the retina for diabetic retinopathy or macular degeneration).

For the management of corneal neovascularization, this would involve perilimbic corneal injection to “protect” the cornea from the advancing blood vessels. This method may also be utilized shortly after a corneal insult in order to prophylactically prevent corneal neovascularization. In this situation the antimicrotubule agent could be injected in the perilimbic cornea interspersed between the corneal lesion and its undesired potential limbic blood supply. Such methods may also be utilized in a similar fashion to prevent capillary invasion of transplanted corneas. In a sustained-release form injections might only be required 2-3 times per year. A steroid could also be added to the injection solution to reduce inflammation resulting from the injection itself.

Within another aspect of the present invention, methods are provided for treating neovascular glaucoma, comprising the step of administering to a patient a therapeutically effective amount of a protein or polysaccharide containing solubilized anti-microtubule agent to the eye, such that the formation of blood vessels is inhibited. Briefly, neovascular glaucoma is a pathological condition wherein new capillaries develop in the iris of the eye. The angiogenesis usually originates from vessels located at the pupillary margin, and progresses across the root of the iris and into the trabecular meshwork. Fibroblasts and other connective tissue elements are associated with the capillary growth and a fibrovascular membrane develops which spreads across the anterior surface of the iris. Eventually this tissue reaches the anterior chamber angle where it forms synechiae. These synechiae in turn coalesce, scar, and contract to ultimately close off the anterior chamber angle. The scar formation prevents adequate drainage of aqueous humor through the angle and into the trabecular meshwork, resulting in an increase in intraocular pressure that may result in blindness.

Neovascular glaucoma generally occurs as a complication of diseases in which retinal ischemia is predominant. In particular, about one third of the patients with this disorder have diabetic retinopathy and 28% have central retinal vein occlusion. Other causes include chronic retinal detachment, end-stage glaucoma, carotid artery obstructive disease, retrolental fibroplasia, sickle-cell anemia, intraocular tumors, and carotid cavernous fistulas. In its early stages, neovascular glaucoma may be diagnosed by high magnification slitlamp biomicroscopy, where it reveals small, dilated, disorganized capillaries (which leak fluorescein) on the surface of the iris. Later gonioscopy demonstrates progressive obliteration of the anterior chamber angle by fibrovascular bands. While the anterior chamber angle is still open, conservative therapies may be of assistance. However, once the angle closes surgical intervention is required in order to alleviate the pressure.

Therefore, within one embodiment of the invention, the polysaccharide containing solubilized anti-microtubule compositions described herein can be administered (e.g., topically) to the eye in order to treat early forms of neovascular glaucoma. Within other embodiments of the invention, the compositions described herein can be implanted by injection of the composition into the region of the anterior chamber angle. This provides a sustained localized increase of antimicrotubule agents, and prevents vascular fibroproliferative tissue growth into the area. Implanted or injected antimicrotubule compositions which are placed between the advancing capillaries of the iris and the anterior chamber angle can “defend” the open angle from fibrovascular tissue growth. Within other embodiments, the polysaccharide containing solubilized anti-microtubule compositions may also be placed in any location such that the anti-microtubule agent is continuously released into the aqueous humor. This would increase the anti-mictotubule agent concentration within the humor, which in turn bathes the surface of the iris and its abnormal fibrovascualr tissue thereby providing another mechanism by which to deliver the medication. These therapeutic modalities may also be useful prophylactically and in combination with existing treatments.

Within another aspect of the present invention, methods are provided for treating proliferative diabetic retinopathy, comprising the step of administering to a patient a therapeutically effective amount of a composition as described herein to the eyes, such that the formation of blood vessels is inhibited.

Briefly, the pathology of diabetic retinopathy is thought to be similar to that described above for neovascular glaucoma. In particular, background diabetic retinopathy is believed to convert to proliferative diabetic retinopathy under the influence of retinal hypoxia. Generally, neovascular tissue sprouts from the optic nerve (usually within 10 mm of the edge), and from the surface of the retina in regions where tissue perfusion is poor. Initially the capillaries grow between the inner limiting membrane of the retina and the posterior surface of the vitreous. Eventually, the vessels grow into the vitreous and through the inner limiting membrane. As the vitreous contracts, traction is applied to the vessels, often resulting in shearing of the vessels and blinding of the vitreous due to hemorrhage. Fibrous traction from scarring in the retina may also produce retinal detachment.

The conventional therapy of choice is panretinal photocoagulation to decrease retinal tissue, and thereby decreasing retinal oxygen demands. Although initially effective, there is a high relapse rate with new lesions forming in other parts of the retina. Complications of this therapy include a decrease in peripheral vision of up to 50% of patients, mechanical abrasions of the cornea, laser-induced cataract formation, acute glaucoma, and stimulation of subretinal neovascular growth (which can result in loss of vision). As a result, this procedure is performed only when several risk factors are present, and the risk-benefit ratio is clearly in favor of intervention.

Therefore, within particularly preferred embodiments of the invention, proliferative diabetic retinopathy may be treated by injection of a polysaccharide containing solubilized anti-microtubule composition as described herein into the aqueous humor or the vitreous, in order to increase the local concentration of antimicrotubule agent in the retina. Preferably, this treatment should be initiated prior to the acquisition of severe disease requiring photocoagulation.

Within another aspect of the present invention, methods are provided for treating retrolental fibroblasia, comprising the step of administering to a patient a therapeutically effective amount of a polysaccharide containing solubilized anti-microtubule composition as described herein to the eye, such that the formation of blood vessels is inhibited.

Briefly, retrolental fibroblasia is a condition occurring in premature infants who receive oxygen therapy. The peripheral retinal vasculature, particularly on the temporal side, does not become fully formed until the end of fetal life. Excessive oxygen (even levels which would be physiologic at term) and the formation of oxygen free radicals are thought to be important by causing damage to the blood vessels of the immature retina. These vessels constrict, and then become structurally obliterated on exposure to oxygen. As a result, the peripheral retina fails to vascularize and retinal ischemia ensues. In response to the ischemia, neovascularization is induced at the junction of the normal and the ischemic retina.

In 75% of the cases these vessels regress spontaneously. However, in the remaining 25% there is continued capillary growth, contraction of the fibrovascular component, and traction on both the vessels and the retina. This results in vitreous hemorrhage and/or retinal detachment, which can lead to blindness. Neovascular angle-closure glaucoma is also a complication of this condition.

As it is often impossible to determine which cases will spontaneously resolve and which will progress in severity, conventional treatment (i.e., surgery) is generally initiated only in patients with established disease and a well-developed pathology. This “wait and see” approach precludes early intervention, and allows the progression of disease in the 25% who follow a complicated course. Therefore, within one embodiment of the invention, topical administration of polysaccharide containing solubilized anti-microtubule compositions may be accomplished in infants which are at high risk for developing this condition in an attempt to cut down on the incidence of progression of retrolental fibroplasia. Within other embodiments, intravitreous injections and/or intraocular implants of polysaccharide containing solubilized anti-microtubule compositions may be utilized. Such methods are particularly preferred in cases of established disease, in order to reduce the need for surgery.

For paclitaxel, a variety of embodiments are described for the management of fibroproliferative eye diseases. In one preferred embodiment, 0.08-5 mg of paclitaxel is loaded into a micellar carrier incorporated into hyaluronic acid and injected into the eye and releases the drug over a period of time such that the incidence of fibroproliferative eye disease is reduced. In another preferred embodiment, 0.08-5 mg of paclitaxel is applied to the surface of the surgical implant (e.g., artificial lens for cataract surgery, drainage implants for glaucoma filtration surgery, corneal transplant tissue) via the micellar-hyaluronic acid carrier to prevent encapsulation/inappropriate scarring in the vicinity of the implant. In yet another preferred embodiment, a micellar-hyaluronic acid implant containing 0.08-5 mg paclitaxel is applied directly to the surgical site (e.g., into the drainage canal in glaucoma filtration surgery, into the vitreous in cataract surgery, around the cornea in corneal transplant) such that recurrence of inflammation, adhesion formation, or scarring is reduced.

For docetaxel, a variety of embodiments are described for the management of fibroproliferative eye diseases. In one preferred embodiment, 0.05-2.0 mg of docetaxel is loaded into a micellar carrier incorporated into hyaluronic acid and injected into the eye and releases the drug over a period of time such that the incidence of fibroproliferative eye disease is reduced. In another preferred embodiment, 0.05-2.0 mg of docetaxel is applied to the surface of the surgical implant (e.g., artificial lens for cataract surgery, drainage implants for glaucoma filtration surgery, corneal transplant tissue) via the micellar-hyaluronic acid carrier to prevent encapsulation/inappropriate scarring in the vicinity of the implant. In yet another preferred embodiment, a micellar-hyaluronic acid implant containing 0.05-2.0 mg docetaxel is applied directly to the surgical site (e.g., into the drainage canal in glaucoma filtration surgery, into the vitreous in cataract surgery, around the cornea in corneal transplant) such that recurrence of inflammation, adhesion formation, or scarring is reduced.

For vincristine, a variety of embodiments are described for the management of fibroproliferative eye diseases. In one preferred embodiment, 0.01-0.2 mg of vincristine sulfate is loaded into a micellar carrier incorporated into hyaluronic acid and injected into the eye and releases the drug over a period of time such that the incidence of fibroproliferative eye disease is reduced. In another preferred embodiment, 0.01-0.2 mg of vincristine sulfate is applied to the surface of the surgical implant (e.g., artificial lens for cataract surgery, drainage implants for glaucoma filtration surgery, corneal transplant tissue) via the micellar-hyaluronic acid carrier to prevent encapsulation/inappropriate scarring in the vicinity of the implant. In yet another preferred embodiment, a micellar-hyaluronic acid implant containing 0.01-0.2 mg vincristine sulfate is applied directly to the surgical site (e.g., into the drainage canal in glaucoma filtration surgery, into the vitreous in cataract surgery, around the cornea in corneal transplant) such that recurrence of inflammation, adhesion formation, or scarring is reduced.

For vinblastine, a variety of embodiments are described for the management of fibroproliferative eye diseases. In one preferred embodiment, 0.05-1.0 mg of vinblastine sulfate is loaded into a micellar carrier incorporated into hyaluronic acid and injected into the eye and releases the drug over a period of time such that the incidence of fibroproliferative eye disease is reduced. In another preferred embodiment, 0.05-1.0 mg of vinblastine sulfate is applied to the surface of the surgical implant (e.g., artificial lens for cataract surgery, drainage implants for glaucoma filtration surgery, corneal transplant tissue) via the micellar-hyaluronic acid carrier to prevent encapsulation/inappropriate scarring in the vicinity of the implant. In yet another preferred embodiment, a micellar-hyaluronic acid implant containing 0.05-1.0 mg vinblastine sulfate is applied directly to the surgical site (e.g., into the drainage canal in glaucoma filtration surgery, into the vitreous in cataract surgery, around the cornea in corneal transplant) such that recurrence of inflammation, adhesion formation, or scarring is reduced.

For cholchicine, a variety of embodiments are described for the management of fibroproliferative eye diseases. In one preferred embodiment, 0.05-1 mg of cholchicine is loaded into a micellar carrier incorporated into hyaluronic acid and injected into the eye and releases the drug over a period of time such that the incidence of fibroproliferative eye disease is reduced. In another preferred embodiment, 0.05-1 mg of cholchicine is applied to the surface of the surgical implant (e.g., artificial lens for cataract surgery, drainage implants for glaucoma filtration surgery, corneal transplant tissue) via the micellar-hyaluronic acid carrier to prevent encapsulation/inappropriate scarring in the vicinity of the implant. In yet another preferred embodiment, a micellar-hyaluronic acid implant containing 0.05-1 mg cholchicine is applied directly to the surgical site (e.g., into the drainage canal in glaucoma filtration surgery, into the vitreous in cataract surgery, around the cornea in corneal transplant) such that recurrence of inflammation, adhesion formation, or scarring is reduced.

It should be readily evident to one of skill in the art that any of the previously mentioned anti-microtubule agents, or derivatives and analogues thereof, can be utilized to create variation of the above compositions without deviating from the spirit and scope of the invention.

### EXAMPLES

### Example 1

### Production of a Micellar Carrier for Paclitaxel Dispersal

A micellar carrier for paclitaxel was prepared as follows. A 60:40 methoxy polyethylene glycol (MePEG):poly(DL-lactide) diblock copolymer was prepared by combining 60 g of DL-lactide and 40 g of MePEG (MW=2,000 g/mol) in a round bottom glass flask containing a Teflon™-coated stir bar. The mixture was heated to 140° C. with stirring in a temperature controlled mineral oil bath until the components melted to form a homogeneous liquid. Then 0.1 g (or 0.5 g in some batches) of stannous 2-ethyl hexanoate was added to the molten mixture and the reaction was continued for 6 hours at 140° C. with continuous stirring. The reaction was terminated by cooling the product to ambient temperature. The product, 60:40 MePEG:poly(DL-lactide) diblock copolymer, was stored in sealed containers at 2-8° C. until use.

### Example 2

### Paclitaxel Dispersed in a Micellar Carrier to Make a 150 mg Vial Formulation

Paclitaxel was dispersed in the micellar carrier from Example 1 as follows. Reaction glassware washed and rinsed with Sterile Water for Irrigation USP, and dried at 37° C., followed by depyrogenation at 250° C. for at least 1 hour. First, a phosphate buffer (0.08 M, pH 7.6) was prepared. The buffer was dispensed at the volume of 10 ml per vial. The vials were heated for 2 hours at 90° C. to dry the buffer. The temperature was then raised to 160° C. and the vials dried for an additional 3 hours.

The polymer micelles (from Example 1) were dissolved in acetonitrile at 15% w/v concentration with stirring and heat. The polymer solution was then centrifuged at 3000 rpm for 30 minutes. The supernatant was poured off and set aside. Additional acetonitrile was added to the precipitate and centrifuged a second time at 3000 rpm for 30 minutes. The second supernatant was pooled with the first supernatant. Paclitaxel was weighed and then added to the supernatant pool. The solution was brought to the final desired volume with acetonitrile.

The solution containing paclitaxel dispersed in the polymer-based micelles was dispensed into vials containing previously dried phosphate buffer at a volume of 10 ml per vial. The vials were then vacuum dried to remove the acetonitrile. The micellar paclitaxel was then terminally sterilized by irradiation with at least 2.5 MRad Cobalt-60 (Co-60) γ-rays.

### Example 3

### Paclitaxel Dispersed in a Micellar Carrier to Make an 11 mg Vial Formulation

Paclitaxel was dispersed into the micellar carrier from Example 1 as follows. Reaction glassware washed and rinsed with Sterile Water for Irrigation USP, dried at 37° C., followed by depyrogenation at 250° C. for at least 1 hour. First, a phosphate buffer, 0.08M, pH 7.6 is prepared. The buffer is dispensed at the volume of 1 mL per vial. The vials are heated for 2 hours at 90° C. to dry the buffer. The temperature is then raised to 160° C. and the vials are dried for an additional 3 hours.

The polymer was dissolved in acetonitrile at 10% w/v concentration with stirring and heat. The polymer solution was then centrifuged at 3000 rpm for 30 minutes. The supernatant was poured off and set aside. Additional acetonitrile was added to the precipitate and centrifuged a second time at 3000 rpm for 30 minutes. The second supernatant was pooled with the first supernatant. Paclitaxel was weighed and then added to the supernatant pool. The solution was brought to the final desired volume with acetonitrile to make a 9.9% polymer solution containing 1.1% paclitaxel.

To manufacture development batches of final product vials, the micellar paclitaxel was dispensed into the vials containing dried phosphate buffer at a volume of 1 ml per vial. The vials were placed in a vacuum oven at 50° C. The vacuum was set at □−80 kPa and the vials remained in the oven overnight (15 to 24 hours). The vials were plugged with Teflon™-faced gray butyl stoppers and sealed with aluminum seals. The solution containing paclitaxel dispersed in the polymer-based micelles was sterilized using 2.5 MRad γ radiation. Each vial contains approximately 11 mg paclitaxel, 99 mg polymer, and 11 mg phosphate salts. The vials were used or stored at 2 to 8° C. until constitution.

### Example 4

### Paclitaxel Dispersed in a Microemulsion in a Hyaluronic Acid Gel

Paclitaxel in a microemulsion carrier was incorporated into a hyaluronic acid gel as follows. Forty grams of water was added to a beaker that contained 1 g hyaluronic acid (180 kDa, Bioiberica, Barcelona, Spain). The mixture was allowed to dissolve with stirring (400 rpm for at least 30 minutes) to form a homogeneous gel. To 38.5 g of Labrasol® (caprylocaproyl macrogol-8 glycerides) was added 100 mg of paclitaxel and the mixture stirred (400 rpm for at least 20 minutes) until a clear solution formed. To the paclitaxel solution was added 5 g of Labrafac® CC (medium chain triglycerides, C8-C10 fatty acids) and 16.5 g Plurol® Oleique (polyglyceryl-6 dioleate) with continued stirring for at least 10 minutes to form a visibly homogeneous mixture. The paclitaxel phase was added to the hyaluronic acid phase with further stirring for at least one hour. After stirring, the composition was allowed to stand for at least one hour to allow most of the bubbles to migrate from the gel. The product contains about 0.99 mg paclitaxel/g gel and 9.9 mg hyaluronic acid/g gel.

This composition is alternately prepared with hyaluronic acid having a molecular weight of 1 MDa (Genzyme, Cambridge, Mass.). In these compositions, the exact process is duplicated with the exception that longer stirring times and standing times are used for phases containing higher molecular weight hyaluronic acid. Typically, these are increased by a factor of 5 to 10. Following stirring, if a homogeneous phase is not formed, the mixture is transferred to a 100 ml syringe, attached to a second 100 ml syringe, and then transferred back and forth 30 times between the two syringes through a 1/16″ ID tube to effect mixing. Following that, the mixture is allowed to stand for about 16 hours.

### Example 5

### Paclitaxel Dispersed in a Micellar Carrier in a Hyaluronic Acid Hydrogel

Paclitaxel dispersed in a micellar carrier was incorporated into a hyaluronic acid hydrogel as follows. Two milliliters sterile saline were added to a vial that contained approximately 11 mg paclitaxel, 99 mg polymer, and 11 mg phosphate salts (prepared according to Example 3). The contents of the vial were dissolved by placing the vial in a water bath at 37° C. for approximately 30 minutes with periodic vortexing. Using a 1 ml syringe, a 0.82 ml aliquot of the micellar paclitaxel solution was withdrawn from the vial and was injected into 22.5 ml hyaluronic acid gel (INTERGEL®, Ethicon, Inc., Sommerville, N.J.). The sample was mixed to produce a homogeneous solution of paclitaxel dispersed in micelles (i.e., micellar paclitaxel) in a hyaluronic acid gel.

### Example 6

### Paclitaxel Dispersed in a Micellar Carrier into 1 MDa Hyaluronic Acid Hydrogels

A micellar paclitaxel composition was prepared from the copolymer prepared according to Example 1 as follows. A solid composition capable of forming micelles upon constitution with an aqueous medium was prepared as follows. Then 41.29 g of MePEG (MW=2,000 g/mol) was combined with 412.84 g of 60:40 MePEG:poly(DL-lactide) diblock copolymer (see, e.g., Example 1) in a stainless steel beaker, heated to 75° C. in a mineral oil bath and stirred by an overhead stirring blade. Once a clear liquid was obtained, the mixture was cooled to 55° C. To the mixture was added a 200 ml solution of 45.87 g paclitaxel in tetrahydrofuran. The solvent was added at approximately 40 ml/min and the mixture stirred for 4 hours at 55° C. After mixing for this time, the liquid composition was transferred to a stainless steel pan and placed in a forced air oven at 50° C. for about 48 hours to remove residual solvent. The composition was then cooled to ambient temperature and was allowed to solidify to form a paclitaxel-polymer matrix.

A 2 g aliquot of paclitaxel-polymer matrix was dissolved in 100 ml water and the pH adjusted to between 6 and 8 by the addition of 1 M sodium hydroxide solution. Into a separate container, 1 mg of 1 MDa hyaluronic acid (Genzyme, Cambridge, Mass.) was added and then 1 ml of the pH adjusted paclitaxel solution was added with stirring to dissolve the hyaluronic acid. The result was a hyaluronic acid gel containing 10 mg/ml hyaluronic acid and 2 mg/ml paclitaxel. A second formulation was prepared in a similar manner to a concentration of 15 mg/ml paclitaxel by dissolving 15 g of paclitaxel-polymer matrix in 100 ml prior to pH adjustment.

### Example 7

### Preparation of Non-Crosslinked Hydroxypropylcellulose Films with Paclitaxel

Five grams of ethyl cellulose and hydroxypropyl cellulose (or other cellulose) with a ratio from 100:0 to 0:100 were dissolved in 100 ml of acetone. Then 5-500 mg of paclitaxel were added and completely dissolved in the acetone solution. The cellulose/acetone/paclitaxel solution was cast onto the release liner using a casting knife with 40 mil opening. The dried cellulose film was obtained after the evaporation of acetone. The samples were further dried in vacuum oven overnight.

### Example 8

### Preparation of Crosslinked Hydroxypropylcellulose Films with Paclitaxel

Five grams of ethyl cellulose and hydropropyl cellulose (or other cellulose) with a ratio from 100:0 to 0:100 were dissolved in 95 ml of acetone. Then 5-500 mg of paclitaxel were added and completely dissolved in the acetone solution. Then 4 ml of acetic acid solution (5%) was added into the solution to make the above solution pH around 2 to 3. Also, 1 ml of 5% glutaraldehyde solution was added into the above solution. The cellulose/acetone/paclitaxel solution was cast onto the release liner using a casting knife with 40 mil opening. The dried cellulose film was obtained after the evaporation of acetone. The samples were further dried in vacuum oven overnight.

### Example 9

### Anti-Microtubule Agent-Loaded Non-Cross-Linked Polymeric Films Composed of Chitosan

Five grams of chitosan (Aldrich)/glycerol (Aldrich) was dissolved in 100 ml of 5% aqueous acetic acid solution. The ratio between chitosan and glycerol is 70:30. The solution was stirred at 600 rpm until the chitosan/glycerol was completely dissolved. Then 500 mg of micellar paclitaxel (10% w/w paclitaxel) was added into the above solution. The chitosan solution was stirred until the paclitaxel micelles and chitosan formed a homogenous solution. Each 2 ml of resulting solution was transferred into a 50×9 polystyrene petri dish. The chitosan/glycerol film was formed by evaporating the water completely in a fumehood overnight. The resulting film was soaked in 0.1N NaOH solution for one minute and redried. The film was dried again under vacuum condition (−90 KPa) for at least 24 hours at room temperature.

### Example 10

### Anti-Microtubule Agent-Loaded Cross-Linked Polymeric Films Composed of Chitosan

Five grams of chitosan (Aldrich)/glycerol (Aldrich) was dissolved in 100 ml of 5% aqueous acetic acid solution. The ratio used for chitosan and glycerol is 70:30. The solution was stirred at 600 rpm until the chitosan/glycerol was completely dissolved, and then 500 mg of micellar paclitaxel (10% w/w paclitaxel) was added to the above solution. The mixture was continuously stirred until the paclitaxel containing micelles and chitosan formed a homogenous solution. Then 0.5 ml of 1.0% glutaraldehyde (0.1% in weight percentage relatively to the total sample weight) was added into the above solution, which was then further mixed with a stir bar at 600 rpm for 30 minutes. Two milliliters of the resulting solution was transferred into a 50×9 polystyrene petri dish. The chitosan/glycerol film was formed by evaporating the water completely in fumehood overnight. The film was dried again under vacuum conditions (−90 KPa) for at least 24 hours at room temperature.

### Example 11

### Assessment of Compatibility of a Co-Solvent Carrier with Hyaluronic Acid Hydrogels

Co-solvent systems were prepared by the addition of water miscible organic solvents to a hyaluronic acid (HA) gel containing 20 mg/ml hyaluronic acid in water. The organic solvent was added with stirring in aliquots of 200 μl. After the addition of each aliquot, the mixture was allowed to stir for several minutes and observed for signs of turbidity or rapidly changing viscosity. At the first sign of visually observed turbidity, the volume of organic solvent added was noted and the ratio of co-solvent to water calculated. In the event that turbidity was not observed the maximum amount of solvent added was 5 ml to 2 ml of gel. The results are as follows:

| | Max. amt. added to HA Gel | |
|---|---|---|
| Solvent | without turbidity | |
| N-methylpyrrolidone | >5 | ml in 2 ml |
| Ethoxydiglycol | 2 | ml in 2 ml |
| PEG 200 | >5 | ml in 2 ml |
| Ethanol | 4 | ml in 2 ml |
| Dimethylsulfoxide | >5 | ml in 2 ml |

### Example 12

### Co-Solvent Carrier Suitable for the Incorporation of Paclitaxel into a Hyaluronic Acid Hydrogel

The suitability of a co-solvent carrier for the incorporation of paclitaxel into a hyaluronic acid hydrogel was determined by measuring the maximum solubility of the drug in a co-solvent ratio (solvent:water) that was demonstrated to be compatible with hyaluronic acid as determined in Example 11. After determining suitability in this manner, co-solvent systems may be further assessed in terms of biocompatibility. Solubility was determined as follows:

To a 1 ml aliquot of a co-solvent system described by the ratio of organic solvent to water was added precisely to 5 and 10 mg±10% paclitaxel. The mixtures were equilibrated at room temperature for 16 hours and observed for clarity and particulates in the liquid. Co-solvent mixtures yielding clear, particulate free liquids were described as having a paclitaxel solubility greater than or equal to 5 or 10 mg, respectively, and were considered suitable as carriers for formulations having drug concentrations up to these levels. The results were as follows:

| | Vol. Ratio | | |
|---|---|---|---|
| Co-solvent | (solvent:water) | Solubility | |
| N-methylpyrrolidone | 4.5:2 | >10 | mg/ml |
| Ethoxydiglycol | 1.8:2 | <5 | mg/ml |
| PEG 200 | 4.5:2 | >5, <10 | mg/ml |
| Ethanol | 3.6:2 | >5, <10 | mg/ml |
| Dimethylsulfoxide | 4.5:2 | <5 | mg/ml |

The same suitability assessment may be made for co-solvent ratios capable of dissolving different amounts of drug by changing the mass of drug initially aliquoted at the start of the test. For example, 1 and 3 mg may be tested instead of 5 and 10 mg.

### Example 13

### Preparation of a Co-Solvent/Paclitaxel/Hyaluronic Acid Formulation

A hyaluronic acid hydrogel containing paclitaxel with a co-solvent carrier is prepared as follows. 9 ml of PEG 200 is used to dissolve 30 mg of paclitaxel. Once a clear, particulate free solution results, water is added to adjust the volume to 10 ml. This “active” phase is transferred to a 10 ml syringe. In a second 10 ml syringe, 200 mg of hyaluronic acid (e.g., 1.6M Da molecular weight) is combined with 10 ml of a mixture of PEG 200 and water having a PEG:water ration of 3:7. The powder is allowed to dissolve in the co-solvent mixture over a 16 hour period. If needed to produce a homogeneous solution, the mixture is mixed by transferring it back and forth 30 times between two syringes joined by a short piece of 1/16″ ID tubing. After both syringes are prepared they are connected to a Y-connector, which is connected by its third opening to an empty 20 ml syringe. The two 10 ml syringes are placed in a syringe pump and the contents of both are pumped at the same rate into the 20 ml syringe. Once the transfer is complete the contents of the 20 ml syringe are transferred back and forth 30 times to a second, empty 20 ml syringe attached by a short piece of 1/16″ID tubing. The result is a 20 ml solution that is a hydrogel of hyaluronic acid (10 mg/ml) containing paclitaxel (1.5 mg/ml) in a co-solvent carrier.

### Example 14

### Preparation of 1.5 mg/ml Paclitaxel Polymeric Nanoparticles 1N Hyaluronic Acid Gel

Polymeric paclitaxel nanoparticles can be prepared by methods known in the art, such as the interfacial deposition method as described by Fessi et al., Int. J. Pharm 1989 and specifically for the preparation of paclitaxel-PLGA nanoparticles by Fonseca et al., J. Control. Rel. 2002. Briefly, a 100 mg of PLGA and 1 mg of paclitaxel are dissolved in 10 ml of acetone. An aqueous solution is prepared with 50 mg of Poloxamer™ 188 (which is nonionic polyoxyethylene-polyoxypropylene co-polymer, having a molecular formula of HO(C₂H₄O)ₐ (C₃H₆O)_{b} (C₂H₄O)ₐ H, where a is about 75 and b is about 30 and an average molecular weight of about 8350) in 20 ml of water. Both solutions are stirred until they are free of particulates. The organic solution is added to the aqueous solution and stirred for 15 minutes. The acetone is removed under reduced pressure, and the resulting nanosuspension is filtered through a 1 μm filter. The filtrate is ultracentrifuged twice at 61, 700×g for 1 hour at 4° C. The supernatant is discarded, and the solids are lyophilized for 24 hours. A 3.0 mg/ml paclitaxel solution in water is prepared from the lyophilized nanoparticles. A 20 mg/ml hyaluronic acid gel is prepared in water. The two solutions are blended in equal volumes, and stirred for 30 minutes. The resulting gel has a paclitaxel concentration of 1.5 mg/ml and a hyaluronic acid concentration of 10 mg/ml.

### Example 15

### Nanoparticles of Paclitaxel Contained in a Polysaccharide Gel

An aliquot of nanoparticulate paclitaxel is obtained from its supplier (either commercial or non-commercial) in either an aqueous form or as a lyophilized material for constitution according to the following table.

| Nanoparticle Name | Form | Supplier |
|---|---|---|
| Hydroplex ™ | 10 mg paclitaxel/ml | ImaRx |
| Paclitaxel | solution | |
| Dissocube | 10 mg paclitaxel/ml | SkyePharma PLC |
| Paclitaxel | solution | |
| NanoCrystal ® | 50 mg/ml paclitaxel/ml | Elan Pharmaceuticals |
| Paclitaxel | solution | |

Alternately, NanoCrystal® Paclitaxel is produced using a pearl mill. The milling balls used in such mills range in size from about 0.4 mm to 3.0 mm. Current pearl materials are glass and zirconium oxide. Alternatively, the pearl mills can be made from a hard polymer, e.g., especially cross-linked polystyrene. Depending on the hardness of the drug powder and the required fineness of the particle material, the milling times range from hours to days (Liversidge, in “Drug Nanocrystals for Improved Drug Delivery” at CRS Workshop Particulate Drug Delivery Systems 11-12, Jul. 1996, Kyoto, Japan). The preferred size range for NanoCrystals® is below 400 nm, and about 100 nm for paclitaxel (Liversidge & Cundy, Int J Pharm 1995(125) 91). After the milling process the drug nanoparticles need to be separated from the milling balls.

The aliquot of nanoparticulate paclitaxel is diluted with a 20 mM phosphate buffered 0.9% saline solution to a final concentration of 3 mg paclitaxel/ml. A hyaluronic acid gel phase is prepared by dissolving 20 mg/ml 1 MDa hyaluronic acid (Genzyme, Cambridge, Mass.) in water. A 10 ml aliquot of the gel phase is transferred to a depyrogenated serum bottle and capped with a flat bottomed stopper and sealed. A venting needle is placed in the stopper and the bottle is autoclaved at 135° C. for 15 minutes. After sterilization a 10 ml aliquot of the paclitaxel phase is sterile filtered by passing it through a 0.22 μm filter into the bottle containing the gel. The contents of the bottle are mixed first by inversion of the bottle and finally by repeatedly withdrawing the contents of the bottle through a 25-gauge needle into a syringe and re-injecting the contents into the bottle until a visibly homogeneous liquid is observed. The result is a formulation containing 1.5 mg/ml paclitaxel and 10 mg/ml hyaluronic acid in a sterile buffered aqueous dispersion. The formulation is stored for a maximum of 24 hours at 2-8° C. and may be used by intra-articular injection provided the vial contents are visually clear, with no signs of precipitation. A biocompatible dose may be determined for each formulation using the method described in Example 16.

### Example 16

### Assessment of Biocompatibility of Paclitaxel in a Polysaccharide Formulation

Biocompatibility of paclitaxel formulations given to guinea pigs by intra-articular injection may be assessed as follows. Paclitaxel was incorporated into the test formulation to form a hydrogel by means such as those described in Examples 4, 5, 6, 13, 14, and 15. A 100 μl aliquot was administered by intra-articular injection into the right knee of a healthy male Hartley guinea pig aged at least 6 weeks. After injection, guinea pigs were housed 5 to a cage with free access to food and water. One week after injection, the animals were assessed for swelling, sacrificed, and the knee exposed for visual examination. Visual evidence of swelling or tissue irritation (e.g. fluid, discoloration, and vascularization) indicated a dose dependant response in biocompatibility of the formulation. Swelling greater than 15% increase in joint circumference and evidence of moderate to severe tissue response indicated that the formulation was not biocompatible in the joint after a single injection in the intra-articular model. Absence of these indicators or only a mild response, and swelling less than 15% indicated a biocompatible formulation. Paclitaxel was loaded into a non-polysaccharide micellar carrier and used in this assay of biocompatibility. The results indicated that a 7.5 mg/ml dose of paclitaxel in the micellar carrier was not biocompatible, significant swelling and a moderate tissue response, whereas a 1.5 mg/ml dose of paclitaxel in the micellar carrier was compatible, with only minor swelling or tissue response upon post-mortum examination. Representative data are provided in the following table.

| | | |
|---|---|---|
| | Swelling (% | |
| | Increase in joint | |
| Formulation | circumference | Visual Findings |

Polysaccharide free (control) paclitaxel micelles

| | | |
|---|---|---|
| 1.5 mg/ml paclitaxel | <5% | normal |
| 4.5 mg/ml paclitaxel | >15% | moderate inflammation |
| 7.5 mg/ml paclitaxel | >30% | moderate inflammation |
| 15 mg/ml paclitaxel | >50% | severe inflammation, highly |
| | | vascularized |

Microemulsion formulation (prepared according to Example 4)

| | | |
|---|---|---|
| 1.5 mg/ml paclitaxel | <5% | very mild inflammation |
| 4.5 mg/ml paclitaxel | >15% | moderate inflammation |
| 7.5 mg/ml paclitaxel | >30% | severe inflammation |

Micellar Paclitaxel formulation (prepared according to Example 6)

| | | |
|---|---|---|
| 1.5 mg/ml paclitaxel | <5% | normal |
| 4.5 mg/ml paclitaxel | <5% | normal |
| 7.5 mg/ml paclitaxel | <5% | normal |
| 15 mg/ml paclitaxel | <5% | normal |
| 30 mg/ml paclitaxel | >20% | mild to severe inflammation |

Co-solvent Gel formulation (prepared according to Example 13)

| | | |
|---|---|---|
| 1.5 mg/ml paclitaxel | <5% | normal |
| 4.5 mg/ml paclitaxel | <5% | normal |
| 7.5 mg/ml paclitaxel | >30% | mild inflammation & |
| | | discoloration of infrapatellar |
| | | pad |

### Example 17

### Efficacy of an Anti-Microtubule Agent in a Polysaccharide Matrix Assessed in a Rat Caecal-Sidewall Abrasion Model of Surgical Adhesions

Sprague Dawley rats were prepared for surgery by anesthetic induction with 5% halothane in an enclosed chamber. Animals were transferred to the surgical table, and anesthesia maintained by nose cone on halothane throughout the procedure and Buprenorphen 0.035 mg/kg was injected intramuscularly. The abdomen was shaved, sterilized, draped and entered via a midline incision. The caecum was lifted from the abdomen and placed on sterile gauze dampened with saline. Dorsal and ventral aspects of the caecum were scraped a total of 45 times over the terminal 1.5 cm using a #10 scalpel blade, held at a 45° angle. Blade angle and pressure were controlled to produce punctuated bleeding, while avoiding severe tissue damage or tearing. The left side of the abdominal cavity was retracted and everted to expose a section of the peritoneal wall nearest the natural resting caecal location. The exposed superficial layer of muscle (transverses abdominis) was then excised over an area of 1.0×1.5 cm². Excision included portions of the underlying internal oblique muscle, leaving behind some intact and some torn fibres from the second layer. Minor local bleeding was tamponaded until controlled. The formulations as described in Examples 5 and 6 were deployed at the wounded areas, on the abraded sidewall, between the caecum and sidewall. In addition, any of the other formulations described herein may be deployed in the same manner. The abraded caecum was then positioned over the sidewall wound and sutured at four points immediately beyond the dorsal corners of the wound edge. The large intestine was replaced in a natural orientation continuous with the caecum. The abdominal incision was then closed in two layers with 4-0 silk sutures. Healthy subjects were followed for one week, and then euthanized by lethal injection for post mortem examination to score. Severity of post-surgical adhesions was scored by independently assessing the tenacity and extent of adhesions at the site of caecal-sidewall abrasion, at the edges of the abraded site, and by evaluating the extent of intestinal attachments to the exposed caecum. Adhesions were scored on a scale of 0-4 with increasing severity and tenacity.

### Example 18

### Efficacy of an Anti-Microtubule Agent in a Polysaccharide Matrix Assessed in a Rabbit Uterine Horn Model of Surgical Adhesions

Female New Zealand white rabbits weighing between 3-4 kg were used for surgeries. The animals were acclimated in the vivarium for a minimum of 5 days prior to study initiation and housed individually. Animals were anesthetized by a single injection of ketamine hydrochloride (35 mg/kg) and xylanzine hydrochloride (5 mg/kg). Once sedated, anesthesia was induced with halothane or isofluorane delivered through a mask until the animal was unconscious, when an endotracheal tube was inserted for delivery of halothane or isofluorane to sustain surgical anesthesia. The abdomen was shaved, swabbed with antiseptic, and sterile-draped for surgery. A midline vertical incision 6-7 cm in length was made with a #10 scalpel blade. The uterine horns were brought through the incision and each horn was abraded 20 times in each direction with a #10 scalpel blade held at a 45° angle. A region of the uterine horn, approximately 2 cm in length was abraded along the circumference of the horn, beginning 1 cm from the ovaric end. This injury resulted in generalized erythema without areas of active bleeding. Each side of the abdominal cavity was retracted and everted to expose a section of the peritoneal wall nearest the natural resting location of the horn. The sidewall apposed to the abraded uterine horn was injured by removing a 2.0×0.5 cm² area of the peritoneum. The abraded uterine horn was then positioned over the sidewall wound and sutured at four points of the wound edge. Following completion of the abrasion, before closure, animals were randomized into treatment and non-treatment groups. Treated animals had approximately 1 ml of formulation applied to each horn at the site of attachment to the sidewall. Healthy subjects were followed for one week, and then euthanized by lethal injection for post mortem examination to score the severity of inflammation and adhesions using established scoring systems. Post-surgical adhesions were scored by independently assessing the extent, severity and tenacity of adhesions of each horn to the peritoneal sidewall. Adhesions were scored on a scale of 0-4 depending involvement of the horn in adhesions and a scale of 0-3 with increasing severity and tenacity.

### Example 19

### Efficacy of an Anti-Microtubule Agent in a Polysaccharide Matrix Assessed in a Guinea Pig Model of Osteoarthritis in the Knee

Hartley guinea pigs, at least 6 weeks old, are anesthetized with isoflurane (5% induction-2% maintenance). The knee area on the right leg is shaved and sterilized. A 20 G needle is introduced in the knee joint using a medial approach and the anterior cruciate ligament is cut. This procedure induces osteoarthritic changes in the injured knee detectable 2 weeks after injury and worsening in the following months. Two weeks after the initial procedure, the injured knee is injected with the test formulation as described in Example 13 using a 25 G needle. In addition, any other formulation described herein may also be used in this model. Injection volume is between 0.05 and 0.10 ml. Injections are repeated weekly for a total of 5 injections. Nine weeks following the first intra-articular injection, the animals are sacrificed by cardiac injection of Euthanol. Tissue samples from the knee joint are harvested and prepared for histopathology review. Changes in cellularity, glycosaminoglycan and collagen distribution in the tibial cartilage are assessed. Disease progression is scored and compared to that observed in injured, untreated knee joints.

### Example 20

### Efficacy of an Anti-Microtubule Agent in a Polysaccharide Matrix Assessed in a Mouse Model of a Human Prostate Tumor

Human PC3 prostate cells are maintained in Dubelco's Minimal Essential Medium with 5% fetal calf serum. Male SCID mice are grown to between 25-30 g prior to testing. To test, one million PC3 cells are injected subcutaneously in the flank of SCID mice and tumors allowed to grow until they reach a volume of at least 0.1 cm³. Tumor bearing mice are treated with a 100 μl dose of paclitaxel in a 10 mg/ml hyaluronic acid gel prepared, for example, according to the methods described in Examples 5 and 6. Mice are housed 5 per cage, freely fed food and water, and are assessed bi-weekly for evidence of tumor growth. Tumor size is measured using callipers and measurements of length, width and height of tumor converted to volume using a hemi-ellipsoid formula:
volume=pi/6(length*width*height)

After tumors have progressed beyond 3 cm³, mice are sacrificed by asphyxiation with CO₂. Efficacy is expressed in the ability of the formulation to delay the onset or slow the growth of tumors when data are compared with control data from mice inoculated with tumors not treated with an anti-microtubule agent in a polysaccharide.

### Example 21

### Efficacy of an Anti-Microtubule Agent in a Polysaccharide Matrix Assessed in a Rat Model of Collagen-Induced Rheumatoid-Like Arthritis

Multiple intravenous dosing can be used to evaluate drug efficacy in rats for the treatment of collagen-induced arthritis (CIA), a T-cell dependent model of rheumatoid arthritis. Within approximately two weeks after immunization with type II collagen in Freund's incomplete adjuvant, susceptible rats develop polyarthritis with histologic changes of pannus formation and bone/cartilage erosion. This model is characterized by neovascularization, synovitis and joint destruction within the hind limbs.

Syngeneic female Louvain rats weighing 120-150 g are immunized with native chick type II collagen (CII) to induce CIA. Rats under anesthesia are injected intradermally with 0.5 mg of CII, solubilized in 0.1M acetic acid and emulsified in FIA. Between 90% and 100% of rats typically develop synovitis by day 9 post immunization. At confirmation of arthritis using clinical signs of inflammation, animals are randomly assigned to either one of two drug treatment groups (Dose Level I and Dose Level II) or a control group. Drug-treated groups can be dosed approximately on days 0, 2 and 4, 6, 9, 12 and 15. Animals are euthanized at approximately day 18 following clinical assessment of arthritis.

The degree of clinical arthritis is quantified on a daily basis by an investigator blinded to the study groups, whereby the severity of inflammation of each hind limb is assessed using an integer scale ranging from 0 to 4. This quantification method is based on standardized levels of swelling and peri-articular erythema, with 0 representing normal and 4 representing severe. The sum of the scores for the limbs (maximum number 8) is the arthritis index. An index score between 6 and 8 is considered to represent severe disease.

Hind limb radiographs can be obtained on Day 18 of the treatment schedule and graded according to the extent of soft tissue swelling, joint space narrowing, bone destruction and periosteal new bone formation. An investigator blinded to the treatment protocol should assign radiographic scores. An integer scale of 0 to 3 is used to quantify each hind limb (0=normal, 1=soft tissue swelling, 2=early erosions of bone, 3=severe bone destruction and/or ankylosis). The radiographic joint index is calculated as the sum of both hind limb scores for each rat (maximum possible score of 6).

Sensitization to CII, as measured by anti-CII antibodies on Day 18 can also be determined by standard methods. Histopathological assessment of ankle joints may be conducted using light microscopy under blinded conditions by a pathologist. The animals in the control group typically show marked inflammation involving the joint capsule, cartilage and bone, characteristic of arthritis.

### Example 22

### Clinical Study to Assess Safety and Tolerability of HA-Containing Micellar Paclitaxel for the Treatment of Osteoarthritis

A. Study Design

Patients with a diagnosis of OA of the knee who have failed NSAID therapy are eligible for participation in the study. Seventy-five patients are randomized into the following groups:

| | # of | | Hyaluronic Acid |
|---|---|---|---|
| Treatment | Injections (Weekly) | Paclitaxel Dose | Dose |
| Placebo | 3 | 0 | 0.2 mg in 2 ml |
| Low Dose × 3 | 3 | 25% MTD | 20 mg in 2 ml |
| High Dose × 3 | 3 | 75% MTD | 20 mg in 2 ml |
| Low Dose × 5 | 5 | 25% MTD | 20 mg in 2 ml |
| High Dose × 5 | 5 | 75% MTD | 20 mg in 2 ml |

The MTD (maximum tolerated dose) of paclitaxel given by intra-articular injection is to be determined in a dose escalation phase 1 clinical study involving 20 patients divided into four groups of 5 each receiving hyaluronic acid 20 mg in 2 ml containing paclitaxel in amounts of 0, 1, 5 and 10 mg). In the phase 1 trial, a MTD will be determined as the maximum dose in which the evaluation criteria are met, having minimally acceptable levels of:

(i) pain/discomfort at and after injection

(ii) increased swelling in the joint

(iii) decreased range of motion in the joint

(iv) neutropenia

(v) alopecia

(vi) nausea

(vii) hypersensitivity reaction

(viii) inflammation at the site of injection

After determining the MTD by these means, the clinical test to determine effectiveness of a safe dose may be initiated as follows. After receiving weekly injections according to the table in this example, the patients will be followed by visits at 2, 3, 6 and 12 months after the first treatment. On each treatment day and at each follow-up visit, 5.0 ml of blood 20 ml of urine and 1 ml of synovial fluid are collected and stored frozen. These samples are used to assay markers of disease activity and/or progression by measuring cytokine, metalloproteinase, adhesion molecule and/or growth factor levels.

Dosing schedule may vary by ±1 day and laboratory-testing schedules may vary by ±5 days. After conclusion of treatment, follow-up evaluation visits may occur within ±7 days of the targeted day. The following is a list of samples to be collected from patients for both routine and specialized laboratory tests:

Baseline #1

(i) Chemistry, Hematology, Urinalysis

(ii) ESR

(iii) CRP

(iv) Serum pregnancy test (bHCG)

(v) Radiographs

(vi) Plasma/Serum and Urine Sample

(vii) Each Treatment Day (Day 0, Months 1, 2, 3, 4 and 5)

(viii) Chemistry, Hematology

(ix) ESR

(x) CRP

(xi) Joint Range of Motion

(xii) Joint Swelling

(xiii) Duration of morning stiffness

(xiv) Physician and Patient Global Assessment

(xv) Visual Analog Pain Scale

(xvi) Joint Effusion

(xvii) Plasma/Serum, Urine Sample, Synovial Fluid Sample

B. Evaluation and Testing

Baseline visit #1 will occur at least 28 days prior to the first intra-articular injection to allow for the necessary 1-month washout period if the patient is on other medications (e.g., systemic or intra-articular steroids). If the patient is not on another therapy, then baseline visit #1 will occur at least 10 days prior to the first injection of the test article. A complete medical history and physical examination are obtained as well as urinalysis and screening blood tests, which include: blood chemistries (including liver function tests and creatinine) and hematology (CBC, differential, platelets, Westergren ESR and CRP). Women of childbearing potential must have a negative serum pregnancy test prior to treatment, and should be apprised of the potential risks. Patients whose clinical and laboratory findings fulfill the inclusion criteria are notified and intra-articular injection scheduled.

At baseline visit #1, a physical examination and complete medical history of the patients are done. Interim history and a relevant physical examination of the patients are completed at each treatment day and at 6 and 12 months. At Day 0, all patients will have a thorough clinical evaluation of the knee joint, patient's assessment of pain, patient's global assessment of disease activity and physician's global assessment of disease. At Day 0 and Months 6 and 12, radiographs of affected knee are obtained. Vital signs are obtained prior to dosing. Treatment vital sign monitoring are done at 15-minute intervals post-injection. Patients are treated on Day 0, Months 1, 2, 3, 4 and 5, and follow up visits will occur at Months 6 and 12. In addition, the patients are monitored for safety at 7 days post-infusion. Assessments are completed for both safety and clinical response criteria at each treatment visit and follow-up visit, as defined below.

(i) Chemistry, Hematology

(ii) ESR

(iii) CRP

(iv) Joint Tender

(v) Joint Swelling

(vi) Duration of morning stiffness

(vii) Physician and Patient Global Assessment

(viii) Visual Analog Pain Scale

(ix) Joint Effusion

The patient must be assessed carefully during the first 30 minutes following injection. Vital signs need to be taken at 15-minute intervals and, if stable can be discontinued thereafter.

Adverse events are tabulated and frequencies of events are determined, overall and by dosing group. All events with a WHO Grading of Acute and Subacute Toxicity of Grade 3 or above are tabulated by event, as well as tabulations for all events that have been determined to be possibly or probably related to the test article. Laboratory analyses (chemistries, hematology, synovial fluid analysis) will consist of measurements of change from baseline over time by patient and overall, with plots of actual values compared to normal values for patients by dose group. Logarithmic transformations may be applied as necessary. Group means and standard errors are calculated for the various laboratory parameters. The various Visual Analog Scales are analyzed by computing change from baseline and over time to determine any potential degradation in overall function. Concurrent illnesses are listed and examined as possible confounders in the treatment response relationship. Concurrent medications will also be listed. Effects of previous treatments for OA and any potential related side effects are analyzed and discussed.

Response has been defined by a series of measures related to OA, consisting of the following measures: joint tenderness, joint swelling count, joint effusion, range of motion, morning stiffness, Patient global assessment scale, Physician global assessment scale, Visual Analog Pain Scale. Changes in pain scale, morning stiffness, joint tenderness and joint swelling over time are calculated as change from baseline by dose group and overall. Trend analysis may also be used to assess various parameters over time. Correlations of various measures are performed to determine important and significant responses.

C. Enrollment

Patients enrolled in this study must have OA of the knee confirmed both clinically and radiographically. Patients enrolled in this study must be aged between 21 to 65 years and have failed treatment with at least one NSAID. Patients are eligible for this study if they have no major concurrent illness or laboratory abnormalities and their WBC count>5,000/mm³; Neutrophils>2,500/mm³; Platelet count≧125,000/mm³; hemoglobin≧10 mg/dL; creatinine≦1.4; <2× elevated liver function tests; normal clotting time. Patients must have stable non-steroidal regimen for 1 month prior to study and must discontinue all systemic steroid regimens 1 month prior to study entry. If patients are taking any intra-articular corticosteroids, they must discontinue 1 month prior to study. If the patient is a women of childbearing age, the patient must have a negative serum pregnancy test, and if pre-menopausal and sexually active, using an effective contraceptive.

If the patient has had prior/current treatment with Taxol®, colchicine, alkylating agents or radiation, the patient must not be treated with a paclitaxel/hyaluronic acid preparation. Prior malignancy, major organ allograft, or uncontrolled cardiac, hepatic, pulmonary, renal or central nervous system disease, known clotting deficiency or any illness that increases undue risk to patient will exclude them from this study. Also, if the patient has been treated with an experimental anti-arthritic drug within 90 days of enrollment, the patient must not be treated with a paclitaxel/hyaluronic acid preparation.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A composition comprising a hyaluronic acid or a derivative thereof, an anti-microtubule agent, and a carrier, wherein the anti-microtubule agent is dispersed by the carrier, the carrier is further dispersed in the hyaluronic acid or a derivative thereof, the hyaluronic acid or derivative thereof has a molecular weight of greater than about 900 kDa, the carrier comprises a co-solvent that is miscible with water at a concentration of at least 10% v/v in water, the anti-microtubule agent is soluble in a mixture of water and the co-solvent, and the composition is sterile.

2. The composition of claim 1, wherein the carrier enhances the dispersability of the anti-microtubule agent in an aqueous medium.

3. The composition of claim 1 wherein the hyaluronic acid or derivative thereof is crosslinked.

4. The composition according to claim 1 wherein the composition is in a form selected from a gel, a hydrogel, a film, a paste, a cream, a spray, an ointment, a powder, and a wrap.

5-17. (canceled)

18. The composition of claim 1 wherein the co-solvent is selected from one or more of ethanol, glycerol, ethoxydiglycol, N-methylpyrrolidinone (NMP), polyethyelene glycol (PEG) or a PEG derivative with a molecular weight of up to about 750 g/mol, and dimethylsulfoxide.

19. The composition of claim 18 wherein the co-solvent is selected from one or more of PEG 200, PEG 300, ethanol, ethoxydiglycol, and NMP.

20. The composition according to claim 1 wherein the anti-microtubule agent is selected from taxanes, discodermolide, colchicine, vinca alkaloids, and analogues or derivatives of the taxanes, discodermolide, colchicines, and vinca alkaloids.

21. (canceled)

22. The composition of claim 20 wherein the anti-microtubule agent comprises a taxane, and wherein the taxane is paclitaxel.

23. The composition according to claim 1 in an aqueous solution further comprising at least one of sodium chloride, sodium phosphate salt, monosaccharide, and disaccharide.

24-25. (canceled)

26. The composition according to claim 1 further comprising water.

27. The composition according to claim 1 having a pH in the range of about 4 to about 8.

28-36. (canceled)

37. A diluted composition prepared by the process of combining a composition according to claim 1 with an aqueous solution comprising at least one of sodium chloride, sodium phosphate salt, monosaccharide, and disaccharide.

38. The diluted composition of claim 37 wherein the anti-microtubule agent is present in the diluted composition at a concentration of about 0.01 mg/ml to about 75 mg/ml.

39. The diluted composition of claim 38 wherein the anti-microtubule agent is at a concentration of about 0.1 mg/ml to about 10 mg/ml.

40. The diluted composition of claim 38 wherein the anti-microtubule agent is at a concentration of about 0.1 mg/ml to about 1.5 mg/ml.

41-91. (canceled)

92. A composition comprising:
an anti-microtubule agent;
a first carrier, the first carrier comprising a co-solvent that is miscible with water at a concentration of at least 10% v/v in water, the anti-microtubule agent being soluble in a mixture of water and the co-solvent; and
a second carrier; wherein the anti-microtubule agent is dispersed in the first carrier, and the first carrier is further dispersed in the second carrier, and wherein the second carrier is a polypeptide or polysaccharide, and the composition is sterile.

93. The composition of claim 92 wherein the second carrier is hyaluronic acid or a derivative thereof.

94-97. (canceled)

98. The composition of claim 92 wherein the anti-microtubule agent is a taxanes, discodermolide, colchicine, a vinca alkaloids, or a derivatives thereof.

99. The composition of claim 98 wherein the anti-microtubule agent is paclitaxel or an analog or derivative thereof.

100. The composition of claim 92 wherein the co-solvent is selected from one or more of ethanol, glycerol, ethoxydiglycol, N-methylpyrrolidinone (NMP), polyethyelene glycol (PEG) or a PEG derivative with a molecular weight of up to about 750 g/mol, and dimethylsulfoxide.
